SEQUENCE LISTING (33 sequences)

SEQ ID NO: 1 - protein, 176 aa, Homo sapiens
MRIFAVFIFMTYWHLLNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 2 - protein, 174 aa, Homo sapiens
MACLGFQRHKAQLNLATRTWPCTLLFFLLFIPVFCKAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIAKEKKPSYNRGLCENAPNRARM

SEQ ID NO: 3 - protein, 383 aa, Artificial Sequence
  Description of Artificial Sequence Synthetic polypeptide
MGVLLTQRTLLSLVLALLFPSMASMAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDQEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - DNA, 20 bp, Artificial Sequence
  Description of Artificial Sequence Synthetic primer
gtggtgccgactacaagcga

SEQ ID NO: 5 - DNA, 21 bp, Artificial Sequence
  Description of Artificial Sequence Synthetic primer
tttggaggatgtgccagaggt

SEQ ID NO: 6 - DNA, 20 bp, Artificial Sequence
  Description of Artificial Sequence Synthetic primer
tacccaccgccatactacct

SEQ ID NO: 7 - DNA, 20 bp, Artificial Sequence
  Description of Artificial Sequence Synthetic primer
ctcagggtcttcgtggctca

SEQ ID NO: 8 - DNA, 22 bp, Artificial Sequence
  Description of Artificial Sequence Synthetic primer
atcgagtacatcttcaagccat

SEQ ID NO: 9 - DNA, 22 bp, Artificial Sequence
  Description of Artificial Sequence Synthetic primer
ctttctttggtctgcattcaca

SEQ ID NO: 10 - DNA, 22 bp, Artificial Sequence
  Description of Artificial Sequence Synthetic primer
tctataacgccaacatcgacga

SEQ ID NO: 11 - DNA, 20 bp, Artificial Sequence
  Description of Artificial Sequence Synthetic primer
ttggcgcattcttgaacatg

SEQ ID NO: 12 - DNA, 20 bp, Artificial Sequence
  Description of Artificial Sequence Synthetic primer
agtgcttcgtgcccgatgac

SEQ ID NO: 13 - DNA, 18 bp, Artificial Sequence
  Description of Artificial Sequence Synthetic primer
tgctgcaacacctgtcct

SEQ ID NO: 14 - DNA, 20 bp, Artificial Sequence
  Description of Artificial Sequence Synthetic primer
ccctaggggatgttccagat

SEQ ID NO: 15 - DNA, 20 bp, Artificial Sequence
  Description of Artificial Sequence Synthetic primer
tgaagcttttccctcttcca

SEQ ID NO: 16 - DNA, 21 bp, Artificial Sequence
  Description of Artificial Sequence Synthetic primer
ccgcctgagcaaagtaaatga

SEQ ID NO: 17 - DNA, 19 bp, Artificial Sequence
  Description of Artificial Sequence Synthetic primer
gcaaccgctggtttggatt

SEQ ID NO: 18 - DNA, 23 bp, Artificial Sequence
  Description of Artificial Sequence Synthetic primer
gaggagaaagtggaggtctggtt

SEQ ID NO: 19 - DNA, 21 bp, Artificial Sequence
  Description of Artificial Sequence Synthetic primer
ctctgatgaggaccgcttctg

SEQ ID NO: 20 - DNA, 18 bp, Artificial Sequence
  Description of Artificial Sequence Synthetic primer
gggagcggtgaagatgga

SEQ ID NO: 21 - DNA, 22 bp, Artificial Sequence
  Description of Artificial Sequence Synthetic primer
tcatgttgctcacggaggagta

SEQ ID NO: 22 - DNA, 20 bp, Artificial Sequence
  Description of Artificial Sequence Synthetic primer
agcttggtggtggatgaaac

SEQ ID NO: 23 - DNA, 20 bp, Artificial Sequence
  Description of Artificial Sequence Synthetic primer
ccctcttcagcaaagcagac

SEQ ID NO: 24 - DNA, 19 bp, Artificial Sequence
  Description of Artificial Sequence Synthetic primer
aaggcgcatgtgaactccc

SEQ ID NO: 25 - DNA, 20 bp, Artificial Sequence
  Description of Artificial Sequence Synthetic primer
acggccttgctcttgttttc

SEQ ID NO: 26 - DNA, 19 bp, Artificial Sequence
  Description of Artificial Sequence Synthetic primer
cccagcatctgcaaagctc

SEQ ID NO: 27 - DNA, 20 bp, Artificial Sequence
  Description of Artificial Sequence Synthetic primer
gtcaatgtacagctgccgca

SEQ ID NO: 28 - DNA, 21 bp, Artificial Sequence
  Description of Artificial Sequence Synthetic primer
caactggagcatttactgctg

SEQ ID NO: 29 - DNA, 21 bp, Artificial Sequence
  Description of Artificial Sequence Synthetic primer
tcagttctgtggccttcttgg

SEQ ID NO: 30 - DNA, 19 bp, Artificial Sequence
  Description of Artificial Sequence Synthetic primer
ctgggtttcatccatccga

SEQ ID NO: 31 - DNA, 19 bp, Artificial Sequence
  Description of Artificial Sequence Synthetic primer
tgcggcatcttcaaacctc

SEQ ID NO: 32 - DNA, 19 bp, Artificial Sequence
  Description of Artificial Sequence Synthetic primer
tggaacagccagaaggaag

SEQ ID NO: 33 - DNA, 19 bp, Artificial Sequence
  Description of Artificial Sequence Synthetic primer
agcaggttgtggtggttga

(19) United States
(12) Patent Application Publication
(10) Pub. No.: US 2015/0139994 A1
(43) Pub. Date: May 21, 2015

(54) COMPOSITIONS AND METHODS FOR PREVENTING ALLOGENEIC IMMUNE REJECTION

(71) Applicant: THE REGENTS OF THE UNIVERSITY OF CALIFORNIA, Oakland, CA (US)

(72) Inventor: Yang XU, San Diego, CA (US)

(73) Assignee: THE REGENTS OF THE UNIVERSITY OF CALIFORNIA, Oakland, CA (US)

(21) Appl. No.: 14/539,271

(22) Filed: Nov. 12, 2014

Related U.S. Application Data
(60) Provisional application No. 61/902,983, filed on Nov. 12, 2013.

(51) Int. Cl. C07K 14/705; C12N 5/0735; A61K 38/17; A61K 35/545

(52) U.S. Cl. CPC C07K 14/70521; A61K 35/545; C12N 5/0606; C07K 14/70532; A61K 38/1774; A61K 2035/122
USPC 424/134.1; 435/320.1

(57) ABSTRACT

The present invention provides methods for preventing the allogeneic immune rejection of allogeneic cells, such as cells derived from human Embryonic Stem Cells (hESCs), without suppressing the entire immune system. Also provided a vector containing a CTLA4-Ig and PD-L1 expression cassette, and compositions containing a CTLA4-Ig and PD-L1 for use in preventing allogeneic immune rejection of allogeneic cells.

### CROSS REFERENCE TO RELATED APPLICATION(S)

This application claims the benefit of priority under 35 U.S.C. §119(e) of U.S. Ser. No. 61/902,983, filed Nov. 12, 2013, the entire content of which is incorporated herein by reference.

### GRANT INFORMATION

This invention was made with government support under Grant Nos. CA094254, AI-064569, and AI-045897 awarded by The National Institutes of Health and RM-0173, TR3-05559, and RB4-06244 awarded by the California Institute for Regenerative Medicine, respectively. The United States government has certain rights in the invention.

### BACKGROUND OF THE INVENTION

1. Field of the Invention

The invention relates generally to gene therapy and more specifically to methods and compositions for preventing allogeneic rejection of cells in a host organism without suppressing the entire immune system.

2. Background Information

Human pluripotent stem cells such as human embryonic stem cells (hESCs) can undergo unlimited self-renewal when propagated in the undifferentiated state and retain the pluripotency to differentiate into all three of the primary germ layers: endoderm, mesoderm, and ectoderm and then into all the cell types in the body. Therefore, as a renewable source of various cell types in the body, hESCs hold great promise for the cell replacement therapy of many human diseases. In this context, significant progress has been made in the differentiation of hESCs into various lineages of biologically active cells for cell replacement therapy. However, one major obstacle for clinic development of hESC-based therapy is that the cells derived from the established hESCs will be immune rejected by the allogeneic immune system of the recipients even under chronic immune suppression that itself poses serious risk for cancer and infection. Thus, a need exists for compositions and methods for preventing allogeneic rejection of hESC-derived cells and any other allogeneic cells used for human cell therapy.

### SUMMARY OF THE INVENTION

The present invention is based on the observation that co-expression of PD-L1 and CTLA4-Ig prevent allogeneic rejection of cells derived from human embryonic stem cells (hESCs) in a host organism.

Accordingly, the present invention provides a method of preventing allogeneic rejection of cells derived from pluripotent stem cells including but not limited to human embryonic stem cells (hESCs), human induced pluripotent stem cells (hiPSCs) and any other allogeneic cells in a host organism. The method includes administering to the host organism cells derived from pluripotent stem cells, such as hESCs, wherein the cells are genetically modified with a vector, where the vector includes a polynucleotide encoding CTLA4-Ig, a polynucleotide encoding PD-L1, and a promoter operably linked thereto. As such, allowing expression of CTLA4-Ig and PD-L1 in the host organism prevents allogeneic rejection of the pluripotent stem cell-derived cells such as hESC-derived cells. In various embodiments, the host organism is mammalian, such as a human. In various embodiments, the cells are brown adipocytes, cardiomyocytes, pancreatic beta cells, cartilage or bone-forming cells, or vascular cells.

In another aspect, the present invention provides an expression cassette comprising a promoter functionally linked to a polynucleotide encoding CTLA4-Ig and a polynucleotide encoding PD-L1. Also provided are a vector, such as a bacterial artificial chromosome (BAC)-based targeting vector, that includes the expression cassette.

In yet another aspect, the present invention provides a method of preventing allogeneic rejection of cells derived from pluripotent stem cells including but not limited to human embryonic stem cells (hESCs), human induced pluripotent stem cells (hiPSCs) and any other allogeneic cells in a host organism. The method includes administering to the host organism in need thereof a therapeutically effect amount of CTLA4-Ig and PD-L1. In various embodiments, the CTLA4-Ig and PD-L1 may be administered alone or in combination with hESC-derived cells or other allogeneic cells, such that allogeneic rejection of the hESC-derived cells or other allogeneic cells, is reduced and/or inhibited. In various embodiments, the host organism is mammalian, such as a human. In various embodiments, the cells are brown adipocytes, cardiomyocytes, pancreatic beta cells, cartilage or bone-forming cells, or vascular cells.

In yet another aspect, the invention provides a pharmaceutical composition. The composition includes CTLA4-Ig and PD-L1, and may be administered alone or in combination with pluripotent stem cell-derived cells such as hESC-derived cells or other allogenic cells.

### BRIEF DESCRIPTION OF THE DRAWINGS

FIGS. 1A-1C are a series of pictorial and graphical diagrams showing generation of hESCs with PD-L1 and CTLA4-Ig expression cassette targeted at HPRT1 locus. FIG. 1A shows the endogenous human HPRT1 locus on chromosome X. Open box indicates the 3′ UTRs of HPRT1. Filled boxes indicate part of HPRT1 coding sequence. Exon 9 (e9) is the last exon. The stop codon (TAA) and the binding sites of the primers used for identification of targeting clones are indicated. FIG. 1B shows a BAC-based targeting vector. FIG. 1C shows the results from a flow cytometry assay of the cell membrane expression of PD-L1.

FIGS. 2A-2E are pictorial diagrams showing that the combination of PD-L1 and CTLA4-Ig protects hESC-derived cells such as teratomas from allogeneic rejection. FIG. 2A shows the results from FACS analysis of representative spleen and transplanted human thymus taken from humanized mice. FIG. 2B shows that extensive T cell infiltration was detected in the teratomas formed by WT hESCs, but not the ones formed by C4IP-hESCs in Humanized mice. FIG. 2C shows that cell necrosis was detected in the teratomas derived from WT hESCs in humanized mice. Cell morphology was revealed by heamatoxylin and eosin staining. FIG. 2D shows a summary of teratoma formation, immune rejection and CD4+ T cell infiltration. FIG. 2E is a graphical diagram showing relative mRNA levels of IL-10, TGFβ1, and IL-2 in T cells isolated from CP hESC- and ET hESC-derived teratomas formed in the same Hu-mouse, determined by real-time PCR.

FIG. 3 is a graphical diagram showing a comparison of the expressions of the marker genes of the three germ layers in C4IP-hESCs derived teratomas taken from NSG and humanized mice. Mean values are presented with SD (for NSG mice, n=7; for hu-mice, n=12).

FIG. 4 is a graphical diagram showing that a combination of PD-L1 and CTLA4-Ig protects hESCs derived neural progenitor cells (NPCs) from allogeneic rejection. Expression of NPCs marker genes NESTIN and SOX2 in C4IP-hESCs derived NPCs were revealed by immunostaining and relative mRNA levels of the neuroepithelial markers SOX2, SOX1 and PAX6, and PD-L1 and CTLA4-Ig in NPCs were revealed by real-time PCR and compared to those in WT hESCs. T cell infiltration was detected in WT NPC transplants but not in C4IP NPC transplants in humanized mice. T cells were identified by CD3/CD4 and CD3/CD8 double staining. Human cells in the grafts were identified by a human nuclei specific antibody (Hu-Nuclei). WT and C4IP NPC transplants in NSG mice were stained as negative controls, while the spleen from a humanized mouse was stained as a positive control. Nuclei were counterstained with DAPI.

FIGS. 5A-5C are a series of pictorial diagrams showing that neither PD-L1 nor CTLA4-Ig alone can protect the derivatives of hESCs. FIG. 5A shows that T cell infiltration was detected in the teratomas formed by WT hESCs, PD-L1-KI-hESCs and CTLA4-Ig-KI-hESCs in Humanized mice. T cells were identified by CD4, CD8 and CD3 antibodies. FIG. 5B shows that cell necrosis was detected in the teratomas derived from WT hESCs, PD-L1-KI-hESCs and CTLA4-Ig-KI-hESCs in humanized mice. Cell morphology was revealed by heamatoxylin and eosin staining. FIG. 5C shows a summary of teratoma formation, tumor rejection and CD4+ T cell infiltration.

FIGS. 6A-6C are a series of pictorial diagrams showing characterization of CP-hESCs (Clone C4IP-1 and -2). FIG. 6A shows the results from quantitative real-time PCR analysis of the mRNA levels of the pluripotency genes in WT and C4IP hESCs. FIG. 6B shows surface expression of the human ESC specific markers by flow cytometry. FIG. 6C shows that C4IP-hESCs form well-differentiated teratomas in SCID mice.

FIGS. 7A-7D are a series of pictorial and graphical diagrams showing that the combination of PD-L1 and CTLA4-Ig protects HUES-8, another human embryonic stem cell line, derived teratomas from allogeneic rejection. FIG. 7A shows that the expression, secretion and dimerization of CTLA4-Ig were analyzed by western blotting, and cell membrane expression of PD-L1 was analyzed by flow cytometry assay. FIG. 7B shows that T cell infiltration was detected in the teratomas formed by WT HUES-8 but not the ones formed by C4IP-HUES-8 in Humanized mice. FIG. 7C shows that cell necrosis was detected in the teratomas derived from WT HUES-8 in humanized mice. FIG. 7D shows a summary of teratoma formation, teratomas rejection and CD4+ T cell infiltration.

FIGS. 8A-8D are pictorial diagrams showing characterization of CP hESC-derived fibroblasts and cardiomyocytes. FIG. 8A shows the relative mRNA levels of β2-microglobulin and HLA class II (DQB1) in hESC, fibroblast (-F), cardiomyocyte (-C), human adult heart tissue and teratoma (-T) were determined by real-time PCR. Mean values are presented with SD (n=3). FIG. 8B shows the expression, secretion and dimerization of CTLA4-Ig in CP-F as analyzed by Western blotting. FIG. 8C shows the relative mRNA levels of the fibroblast-specific genes VIMENTIN and FAP, and PD-L1 and CTLA4-Ig in hESC-derived fibroblasts as determined by real-time PCR. Mean values are presented with SD (n=3). FIG. 8D shows the relative mRNA levels of cardiomyocyte-specific genes NKX2.5, ISL1, MYL7, SIRPA, and PD-L1 and CTLA4-Ig in hESC-derived cardiomyocytes. Mean values are presented with SD (n=3).

FIGS. 9A-9D are a series of pictorial and graphical diagrams showing the characterization of PD-L1-KI and CTLA4-Ig-KI, single knock-in clones in hESCs. FIG. 9A shows that the cell membrane expression of PD-L1 in PD-L1-KI-hESCs was analyzed by flow cytometry assay. FIG. 9B shows that the expression, secretion and dimerization of CTLA4-Ig in CTLA4-Ig-KI-hESCs were analyzed by western blotting. FIG. 9C shows that surface expression of the human ESC specific markers were revealed by flow cytometry. FIG. 9D shows the results from quantitative real-time PCR analysis of the mRNA levels of the pluripotency genes, PD-L1 and CTLA4-Ig in WT, PD-L1-KI and CTLA4-Ig-KI hESCs.

### ABBREVIATIONS

BMP—Bone Morphogenetic Protein
CASM—Coronary artery smooth muscle
cGMP—Current Good Manufacturing Processes
CNS—Central Nervous System
CT—Computed Tomography
CTLA4-Ig—Cytotoxic T lymphocyte antigen 4-immunoglobulin fusion protein
DMEM—Dulbecco's modified Eagle's medium
DMSO—Dimethyl sulphoxide
DPBS—Dulbecco's Phosphate Buffered Saline
EDTA—Ethylenediamine tetraacetic acid
ES Cells—Embryonic stem cells; hESCs are human ES cells. ES cells, including hES cells for the purposes of this invention may be in a naïve state corresponding to ICM cells of the human blastocyst, or the primed state corresponding to flattened epiblast cells (sometimes referred to as “ES-like” cells).
FACS—Fluorescence activated cell sorting
FBS—Fetal bovine serum
GMP—Good Manufacturing Practices
H&E—Hematoxylin & Eosin
hEG Cells—Human embryonic germ cells are stem cells derived from the primordial germ cells of fetal tissue.
hEP Cells—Human embryonic progenitor cells
hiPS Cells—Human induced pluripotent stem cells are cells with properties
similar to hES cells obtained from somatic cells after exposure to hES-specific transcription factors such as SOX2, KLF4, OCT4, MYC, or the genes, RNAs, or proteins encoded by NANOG, LIN28, OCT4, and SOX2.
ICM—Inner cell mass of the mammalian blastocyst-stage embryo.
iPS Cells—Induced pluripotent stem cells are cells with properties similar to ES cells obtained from somatic cells after exposure to ES-specific transcription factors such as SOX2, KLF4, OCT4, MYC, or NANOG, LIN28, OCT4, and SOX2. hiPSCs are human iPS cells.
ITS—Insulin, transferrin, selenium
MEM—Minimal essential medium
PBS—Phosphate buffered saline
PCR—Polymerase Chain Reaction
PD—L1—Programmed death ligand-1
qRT-PCR—quantitative real-time polymerase chain reaction
SFM—Serum-Free Medium

### DETAILED DESCRIPTION OF THE INVENTION

The present invention is based on the observation that co-expression of PD-L1 and CTLA4-Ig prevent allogeneic rejection of cells derived from human embryonic stem cells (hESCs) in a host organism. This observation has been extended to other allogeneic cells.

Before the present compositions and methods are described, it is to be understood that this invention is not limited to particular compositions, methods, and experimental conditions described, as such compositions, methods, and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

The term “comprising,” which is used interchangeably with “including,” “containing,” or “characterized by,” is inclusive or open-ended language and does not exclude additional, unrecited elements or method steps. The phrase “consisting of” excludes any element, step, or ingredient not specified in the claim. The phrase “consisting essentially of” limits the scope of a claim to the specified materials or steps and those that do not materially affect the basic and novel characteristics of the claimed invention. The present disclosure contemplates embodiments of the invention compositions and methods corresponding to the scope of each of these phrases. Thus, a composition or method comprising recited elements or steps contemplates particular embodiments in which the composition or method consists essentially of or consists of those elements or steps.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described.

The term “subject” or “host organism,” as used herein, refers to any individual or patient to which the subject methods are performed. Generally the subject is human, although as will be appreciated by those in the art, the subject may be an animal. Thus other animals, including mammals such as rodents (including mice, rats, hamsters and guinea pigs), cats, dogs, rabbits, farm animals including cows, horses, goats, sheep, pigs, etc., and primates (including monkeys, chimpanzees, orangutans and gorillas) are included within the definition of subject.

The term “therapeutically effective amount” or “effective amount” means the amount of a compound or pharmaceutical composition that will elicit the biological or medical response of a tissue, system, animal or human that is being sought by the researcher, veterinarian, medical doctor or other clinician. Thus, the term “therapeutically effective amount” is used herein to denote any amount of the formulation which causes a substantial improvement in a disease condition when applied to the affected areas repeatedly over a period of time. The amount will vary with the condition being treated, the stage of advancement of the condition, and the type and concentration of formulation applied. Appropriate amounts in any given instance will be readily apparent to those skilled in the art or capable of determination by routine experimentation.

A “therapeutic effect,” as used herein, encompasses a therapeutic benefit and/or a prophylactic benefit as described herein. A prophylactic effect therefore includes delaying or eliminating the onset of allogeneic rejection.

The terms “administration” or “administering” are defined to include an act of providing a compound or pharmaceutical composition of the invention to a subject in need of treatment. The phrases “parenteral administration” and “administered parenterally” as used herein means modes of administration other than enteral and topical administration, usually orally or by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticulare, subcapsular, subarachnoid, intraspinal and infrasternal injection and infusion. The phrases “systemic administration,” “administered systemically,” “peripheral administration” and “administered peripherally” as used herein mean the administration of a compound, drug or other material other than directly into the central nervous system, such that it enters the subject's system and, thus, is subject to metabolism and other like processes, for example, subcutaneous administration.

Thus, the compounds of the invention can be administered in any way typical of such agents for use in preventing or reducing allogeneic rejection of cells in a host organism, or under conditions that facilitate contact of the agents with target cells and, if appropriate, entry into the cells. Entry of a polynucleotide agent into a cell, for example, can be facilitated by incorporating the polynucleotide into a viral vector that can infect the cells.

If a viral vector specific for the cell type is not available, the vector can be modified to express a receptor (or ligand) specific for a ligand (or receptor) expressed on the target cell, or can be encapsulated within a liposome, which also can be modified to include such a ligand (or receptor). A peptide agent can be introduced into a cell by various methods, including, for example, by engineering the peptide to contain a protein transduction domain such as the human immunodeficiency virus TAT protein transduction domain, which can facilitate translocation of the peptide into the cell. In addition, there are a variety of biomaterial-based technologies such as nano-cages and pharmacological delivery wafers (such as used in brain cancer chemotherapeutics) which may also be modified to accommodate this technology.

The viral vectors most commonly assessed for gene transfer to hESCs are based on DNA-based adenoviruses (Ads) and adeno-associated viruses (AAVs) and RNA-based retroviruses and lentiviruses. Lentivirus vectors have been most commonly used to achieve chromosomal integration.

Methods for chemically modifying polynucleotides and polypeptides, for example, to render them less susceptible to degradation by endogenous nucleases or proteases, respectively, or more absorbable through the alimentary tract are well known (see, for example, Blondelle et al., Trends Anal. Chem. 14:83-92, 1995; Ecker and Crook, BioTechnology, 13:351-360, 1995). For example, a peptide agent can be prepared using D-amino acids, or can contain one or more domains based on peptidomimetics, which are organic molecules that mimic the structure of peptide domain; or based on a peptoid such as a vinylogous peptoid. Where the compound is a small organic molecule such as a steroidal alkaloid, it can be administered in a form that releases the active agent at the desired position in the body, or by injection into a blood vessel such that the inhibitor circulates to the target cells.

The compounds of the invention may also be suitably administered by sustained-release systems. Suitable examples of sustained-release compositions include, but are not limited to, semi-permeable polymer matrices in the form of shaped articles, e.g., films, or microcapsules. Sustained-release matrices include polylactides (U.S. Pat. No. 3,773,919, EP 58,481 incorporated herein by reference), copolymers of L-glutamic acid and gamma-ethyl-L-glutamate (U. Sidman et al., Biopolymers 22:547-556 (1983)), poly (2-hydroxyethyl methacrylate) (R. Langer et al., J. Biomed Mater. Res. 15:167-277 (1981), and R. Langer, Chem. Tech. 12:98-105 (1982)), ethylene vinyl acetate (R. Langer et al., Id.) or poly-D-(−)-3-hydroxybutyric acid (EP 133,988). Liposomes containing the compounds of the invention may be prepared by methods known in the art: Epstein, et al., Proc. Natl. Acad. Sci. USA 82:3688-3692 (1985); Hwang et al., Proc. Natl. Acad. Sci. USA 77:4030-4034 (1980); EP 52,322; EP 36,676; EP 88,046; EP 143,949; EP 142,641; U.S. Pat. Nos. 4,485,045 and 4,544,545; and EP 102,324. Ordinarily, the liposomes are of the small (about 200-800 Angstroms) unilamellar type in which the lipid content is greater than about 30 mol. percent cholesterol, the selected proportion being adjusted for the optimal delivery of the compounds of the invention.

In certain embodiments, the invention compounds may further be administered (i.e., co-administered) in combination with an antiinflammatory, antimicrobial, antihistamine, chemotherapeutic agent, antiangiogenic agent, immunomodulator, therapeutic antibody or a neuroprotective agent, to a subject (i.e., host organism) in need of such treatment. Other agents that may be administered in combination with invention compounds include protein therapeutic agents such as cytokines, immunomodulatory agents and antibodies. While not wanting to be limiting, antimicrobial agents include antivirals, antibiotics, anti-fungals and anti-parasitics. When other therapeutic agents are employed in combination with the inhibitors of the present invention they may be used for example in amounts as noted in the Physician Desk Reference (PDR) or as otherwise determined by one having ordinary skill in the art.

The term “co-administer” and “co-administering” and variants thereof refer to the simultaneous presence of two or more active agents in an individual. The active agents that are co-administered can be concurrently or sequentially delivered.

As used herein, the terms “reduce” and “inhibit” are used together because it is recognized that, in some cases, a decrease can be reduced below the level of detection of a particular assay. As such, it may not always be clear whether the expression level or activity is “reduced” below a level of detection of an assay, or is completely “inhibited.” Nevertheless, it will be clearly determinable, following a treatment according to the present methods.

As used herein, an “expression cassette” is made up of one or more genes to be expressed and sequences controlling their expression such as a promoter/enhancer sequence, including any combination of cis-acting transcriptional control elements. The sequences controlling the expression of the gene, i.e., its transcription and the translation of the transcription product, are commonly referred to as regulatory unit. Most parts of the regulatory unit are located upstream of coding sequence of the heterologous gene and are operably linked thereto. The expression cassette may also contain a downstream 3′ untranslated region comprising a polyadenylation site. The regulatory unit of the invention is either directly linked to the gene to be expressed, i.e., transcription unit, or is separated therefrom by intervening DNA such as for example by the 5′-untranslated region of the heterologous gene. Preferably the expression cassette is flanked by one or more suitable restriction sites in order to enable the insertion of the expression cassette into a vector and/or its excision from a vector. Thus, the expression cassette according to the present invention can be used for the construction of an expression vector, in particular a mammalian expression vector.

As used herein, the terms “heterologous coding sequence”, “heterologous gene sequence”, “heterologous gene”, “recombinant gene” or “gene of interest” are used interchangeably. These terms refer to a DNA sequence that codes for a recombinant or heterologous protein product that is sought to be expressed in the mammalian cell and harvested in high amount. The product of the gene can be a protein or polypeptide, but also a peptide. The heterologous gene sequence is naturally not present in the host cell and may be derived from an organism of a different species.

As used herein, the term “genetic modification” is used to refer to any manipulation of an organism's genetic material in a way that does not occur under natural conditions. Methods of performing such manipulations are known to those of ordinary skill in the art and include, but are not limited to, techniques that make use of vectors for transforming cells with a nucleic acid sequence of interest.

The terms “polypeptide,” “peptide,” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, α-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

As used herein, a “promoter” is defined as a regulatory DNA sequence generally located upstream of a gene that mediates the initiation of transcription by directing RNA polymerase to bind to DNA and initiating RNA synthesis.

As used herein, the terms “functionally linked” and “operably linked” are used interchangeably and refer to a functional relationship between two or more DNA segments, in particular gene sequences to be expressed and those sequences controlling their expression. For example, a promoter/enhancer sequence, including any combination of cis-acting transcriptional control elements is operably linked to a coding sequence if it stimulates or modulates the transcription of the coding sequence in an appropriate host cell or other expression system. Promoter regulatory sequences that are operably linked to the transcribed gene sequence are physically contiguous to the transcribed sequence.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The term “antibody” as used herein refers to polyclonal and monoclonal antibodies and fragments thereof, and immunologic binding equivalents thereof. The term “antibody” refers to a homogeneous molecular entity, or a mixture such as a polyclonal serum product made up of a plurality of different molecular entities, and broadly encompasses naturally-occurring forms of antibodies (for example, IgG, IgA, IgM, IgE) and recombinant antibodies such as single-chain antibodies, chimeric and humanized antibodies and multi-specific antibodies. The term “antibody” also refers to fragments and derivatives of all of the foregoing, and may further comprise any modified or derivatised variants thereof that retains the ability to specifically bind an epitope. Antibody derivatives may comprise a protein or chemical moiety conjugated to an antibody. A monoclonal antibody is capable of selectively binding to a target antigen or epitope. Antibodies may include, but are not limited to polyclonal antibodies, monoclonal antibodies (mAbs), humanized or chimeric antibodies, camelized antibodies, single chain antibodies (scFvs), Fab fragments, F(ab′)₂ fragments, disulfide-linked Fvs (sdFv) fragments, for example, as produced by a Fab expression library, anti-idiotypic (anti-Id) antibodies, intrabodies, nanobodies, synthetic antibodies, and epitope-binding fragments of any of the above.

As used herein, the term “humanized mouse” (Hu-mouse) is a mouse developed to carry functioning human genes, cells, tissues, and/or organs. Humanized mice are commonly used as small animal models in biological and medical research for human therapeutics. Immunodeficient mice are often used as recipients for human cells or tissues, because they can relatively easily accept heterologous cells due to lack of host immunity. NSG, or NOD scid gamma (NOD.Cg-Prkdc^{scid} Il2rg^{tm1Wjl}/SzJ), is a strain of inbred laboratory mice that lack mature T cells, B cells, and natural killer (NK) cells. NSG mice are also deficient in multiple cytokine signaling pathways, and they have many defects in innate immunity, which permit the engraftment of a wide range of primary human cells, and enable sophisticated modeling of many areas of human biology and disease.

In addition to previous reports of their capability to mount antigen-specific T cell-dependent antibody responses and mediate xenograft rejection (Lan et al., (2006) Reconstitution of a functional human immune system in immunodeficient mice through combined human fetal thymus/liver and CD34+ cell transplantation, Blood 108, 487-492; Tonomura et al., (2008) Antigen-specific human T-cell responses and T cell-dependent production of human antibodies in a humanized mouse model. Blood 111, 4293-4296), it was shown that Hu-mice could mount effective allogeneic immune rejection of hESC-derived cells. Accordingly, the Hu-mouse model is an excellent in vivo model system for performing a comprehensive analysis of the human immune response against hESC-derived allografts.

As used herein, the term “allogeneic” refers to cells or tissues from individuals belonging to the same species but genetically different, and are therefore immunologically incompatible. Thus, the term “allogeneic cells” refers to cell types that are antigenically distinct, yet belonging to the same species. Typically, the term “allogeneic” is used to define cells, such as stem cells, that are transplanted from a donor to a recipient of the same species.

As used herein, the term “stem cell” generally refers to a cell that on division faces two developmental options: the daughter cells can be identical to the original cell (self-renewal) or they may be the progenitors of more specialized cell types (differentiation). The stem cell is therefore capable of adopting one or other pathway (a further pathway exists in which one of each cell type can be formed). Stem cells are therefore cells which are not terminally differentiated and are able to produce cells of other types.

As used herein, a “pluripotent cell” refers to a cell derived from an embryo produced by activation of a cell containing DNA of all female or male origin that can be maintained in vitro for prolonged, theoretically indefinite period of time in an undifferentiated state, that can give rise to different differentiated tissue types, i.e., ectoderm, mesoderm, and endoderm. “Embryonic stem cells” (ES cells) are pluripotent stem cells derived from the inner cell mass of a blastocyst, an early-stage preimplantation embryo.

A “human embryonic stem cell” (hESC) may therefore be allogeneic when transplanted from a human donor (often a sibling or close relative) to a human recipient. In most cases, the success of allogeneic transplantation depends in part on how well the human leukocyte-associated antigens (HLA) of the donor's stem cells match those of the recipient's stem cells. The higher the number of matching HLA antigens, the greater the chance that the patient's body will accept the donor's stem cells.

An adult stem cell refers to an undifferentiated cell that is found among differentiated cells in a tissue or organ. Exemplary adult stem cells include hematopoietic stem cells, neural stem cells, and mesenchymal stem cells.

The stem cells may be cultured in culture medium comprising conditioned medium, non-conditioned medium, or embryonic stem cell medium. Examples of suitable conditioned medium include IMDM, DMEM, or αMEM, conditioned with embryonic fibroblast cells (e.g., human embryonic fibroblast cells or mouse embryonic fibroblast cells), or equivalent medium. Examples of suitable non-conditioned medium include Iscove's Modified Delbecco's Medium (IMDM), DMEM, or αMEM, or equivalent medium. The culture medium may comprise serum (e.g., bovine serum, fetal bovine serum, calf bovine serum, horse serum, human serum, or an artificial serum substitute) or it may be serum free.

As used herein, “hESC-derived cells” used allogeneically for use in therapy may therefore be any of the diverse somatic cell types derived from the three primary germ layers. Therefore, cells produced using the methods of the present invention that are derivatives of endoderm may be, by way of non-limiting example, hepatocytes (useful in treating degenerative disease of the liver such as cirrhosis); gastrointestinal cells; pancreatic cells such as insulin-secreting islet cells (useful in treating Type I and II diabetes); respiratory cells such as type I and II pneumocytes and other derivatives of endodermal lineage cells. Similarly, derivatives of mesoderm may be skeletal muscle and cardiomyocytes (the latter being useful in treating heart failure and arrhythmias); osteochondral cells (including bone and cartilage of the long bones and axial skeleton such as vertebrae and intervertebral discs (being useful in treating osteoarthritis and degeneration of the intervertebral discs); adipocytes such as brown fat progenitors useful in the treatment of adiposity, coronary disease, hypertension and type I and II diabetes; cutaneous dermal cells useful in promoting scarless wound repair; vascular cells such as vascular endothelium, vascular smooth muscle cells, or vascular pericytes useful in promoting circulation in a tissue and thereby ameliorating the symptoms of ischemic disease; and other derivatives of mesodermal cells. In addition, derivatives of ectoderm may be cells of the central nervous system such as neural progenitors or neurons and glial lineage cells useful in treating spinal cord injury, stroke, Parkinson's disease, and demyelinating diseases; retinal cells such as retinal pigment epithelial cells and retinal neuroglial lineage cells useful in the treatment of age-related macular degeneration and retinitis pigmentosa; and cells derived from the neural crest such as facial cartilage, bone and dermis useful in reconstructive and cosmetic surgery and cells of the peripheral nervous system.

Since the successful establishment of hESCs in 1998 (Thomson, et al., (1998) Embryonic Stem Cell Lines Derived from Human Blastocysts, Science 282, 1145-1147), tremendous progress has been achieved in generating Good Manufacturing Practice (GMP)-grade hESC lines, in large-scale hESC production, and in the lineage-specific differentiation of hESCs (Fu and Xu, (2011) Self-renewal and scalability of human embryonic stem cells for human therapy, Regenerative Medicine 6, 327-334). In addition, the feasibility of hESC-based human cell therapy is further supported by the initiation of two phase I clinic trials of hESC-based cell therapy of spinal cord injury and macular degeneration (Schwartz et al., Embryonic stem cell trials for macular degeneration: a preliminary report, The Lancet 379, 713-720; Wirth Iii, et al., (2011) Response to Frederic Bretzner et al., Target Populations for First-In-Human Embryonic Stem Cell Research in Spinal Cord Injury, Cell Stem Cell 8, 476-478). However, one major hurdle that hinders the clinic development of hESC-based cell therapy is the allogeneic immune rejection of hESC-derived cells by the recipients, even when the cells are transplanted into immune privileged sites due to the breakdown of blood-brain barrier at the lesion site (Boyd et al., (2012) Concise Review: Immune Recognition of Induced Pluripotent Stem Cells, STEM CELLS 30, 797-803). Therefore, to improve the feasibility of hESC-based cell therapy, it is important to develop effective and scalable approaches to protect hESC-derived allografts from immune rejection.

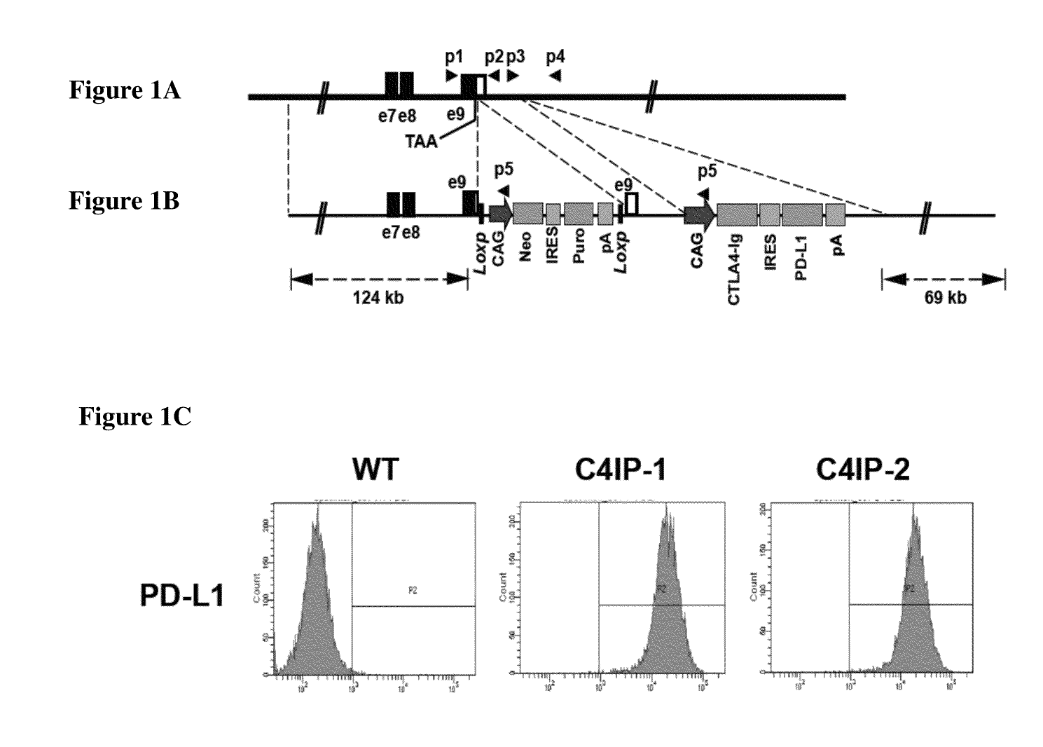

Previous research in transplantation immunology and autoimmunity in mouse models has indicated the critical roles of CTLA4 and PD-L1 in suppressing allogeneic graft rejection and autoimmunity (Fife and Bluestone (2008) Control of peripheral T-cell tolerance and autoimmunity via the CTLA-4 and PD-1 pathways, Immunological Reviews 224, 166-182; Tian, et al., (2007) Induction of Robust Diabetes Resistance and Prevention of Recurrent Type 1 Diabetes Following Islet Transplantation by Gene Therapy, The Journal of Immunology 179, 6762-6769). The data provided herein demonstrates that human cells expressing CTLA4-Ig and PD-L1 elude allogeneic immune rejection. Therefore, to develop a strategy to suppress the allogeneic immune response by disrupting the co-stimulatory pathway and activating the T cell inhibitory pathway, a knock-in strategy to express CTLA4-Ig and PD-L1 in hESC derivatives was designed (FIG. 1A). Thus, knock in hESCs that constitutively express CTLA4-Ig and PD-L1 before and after differentiation are referred to as “CP hESCs”. Using a bacterial artificial chromosome (BAC) based targeting vector that can achieve high efficiency of homologous recombination at the HPRT locus in hESCs (Song, et al., (2010) Modeling Disease in Human ESCs Using an Efficient BAC-Based Homologous Recombination System, Cell Stem Cell 6, 80-89), the CAG-CTLA4-Ig-IRES-PD-L1-PolyA expression cassette was inserted around 200 bp downstream of the HPRT gene (FIG. 1B). The Loxp flanked selection cassette was inserted between the stop codon and the polyA signal sequence of HPRT1 to block its expression, introducing both positive and negative selections during targeting process. The CAG promoter driving expression cassette, CAG/CTLA4-Ig/IRES/PD-L1/pA, was inserted about 600 bps downstream of HPRT1 gene. The sizes of homologous arms are indicated. IRES, internal ribosomal entry site (FIG. 1B). Because hypoxanthine phosphoribosyltransferase 1 (HPRT1) is a X-linked gene, the homologous recombination between the targeting vector and the endogenous HPRT locus in male hESCs led to the suppression of HPRT gene expression, which could be easily selected by their insensitivity to 6-TG. Cells were seeded onto 12-well plates, the next day the media were changed to that containing hypoxanthine/aminopterin/thymidine (HAT), or 6-thioguanine (6-TG), or without a drug. After being treated for three days, the cells were stained with an alkaline phosphatase detection kit.

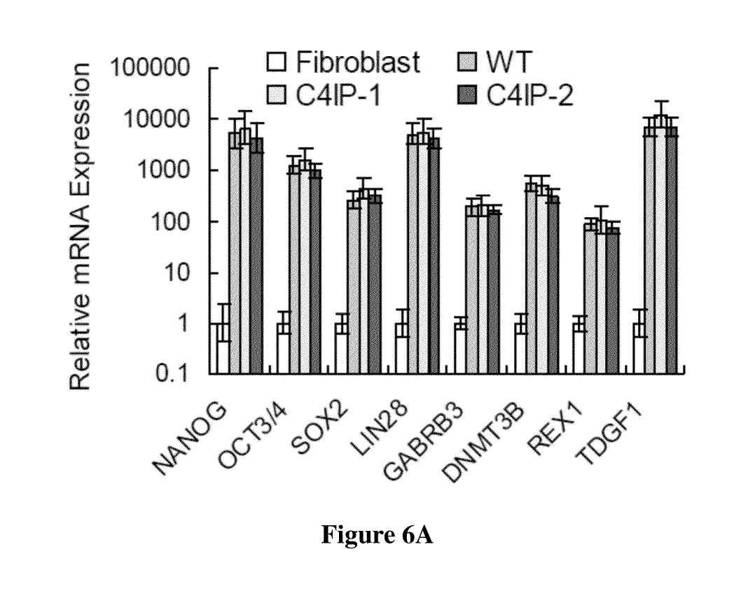

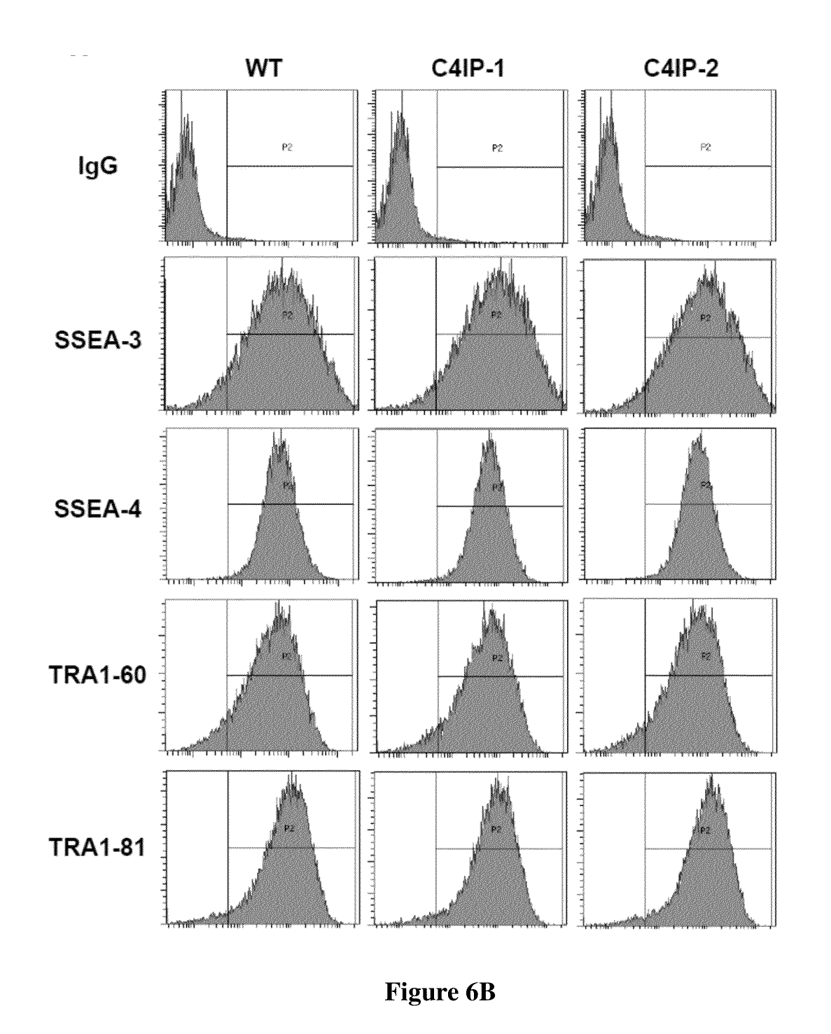

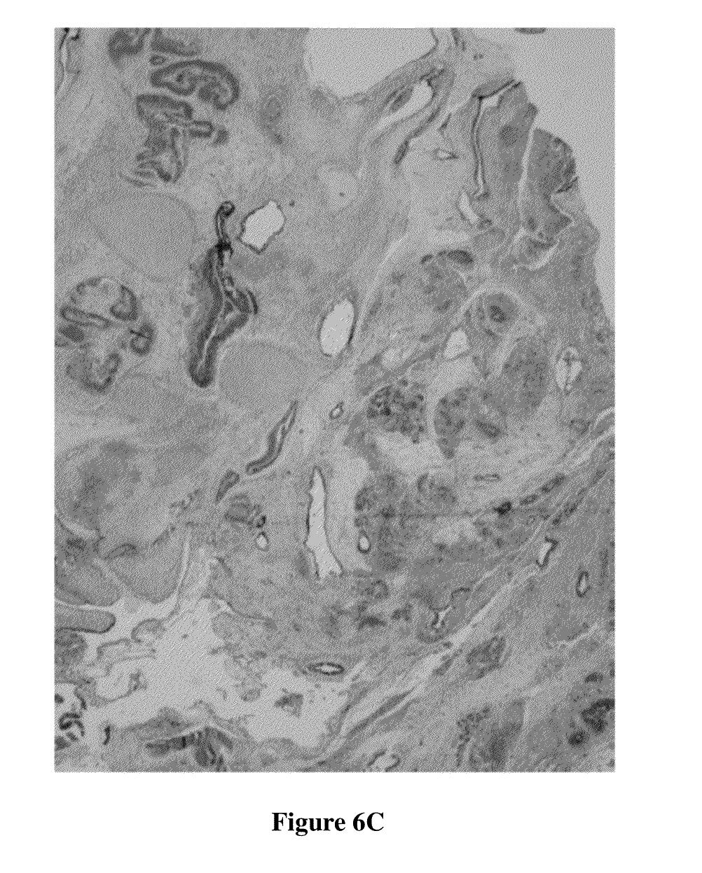

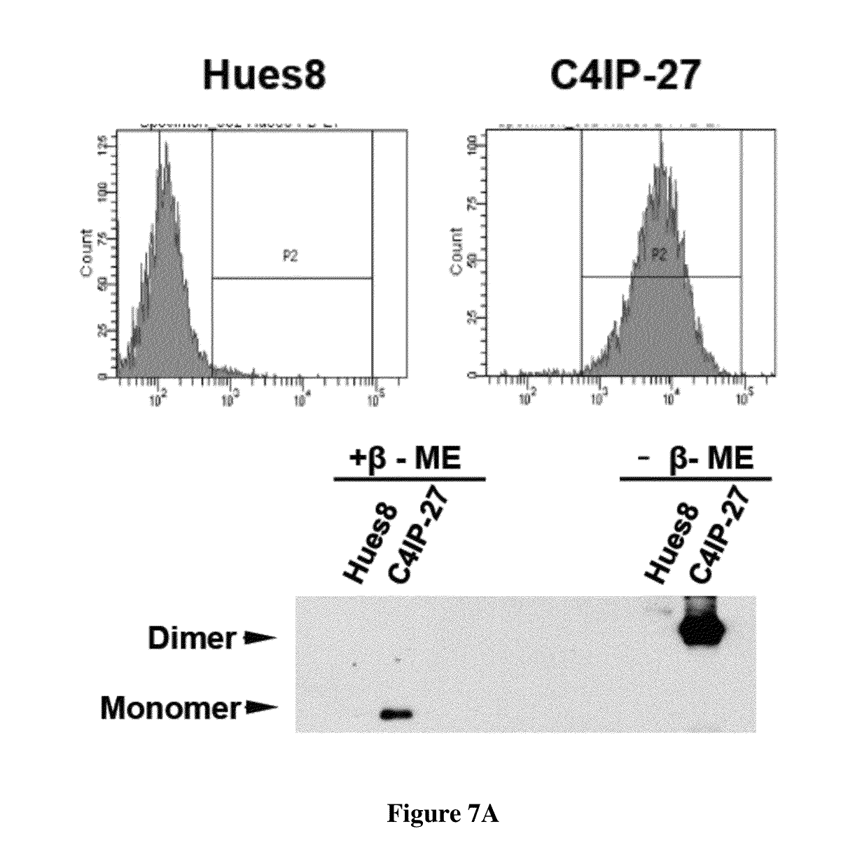

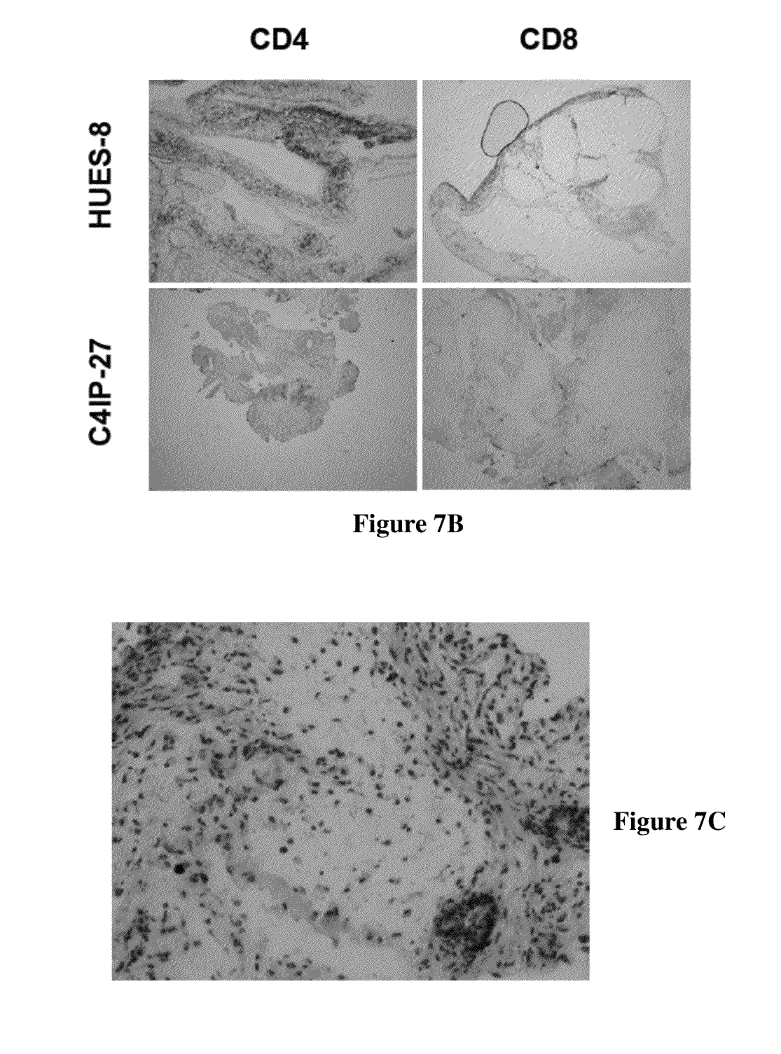

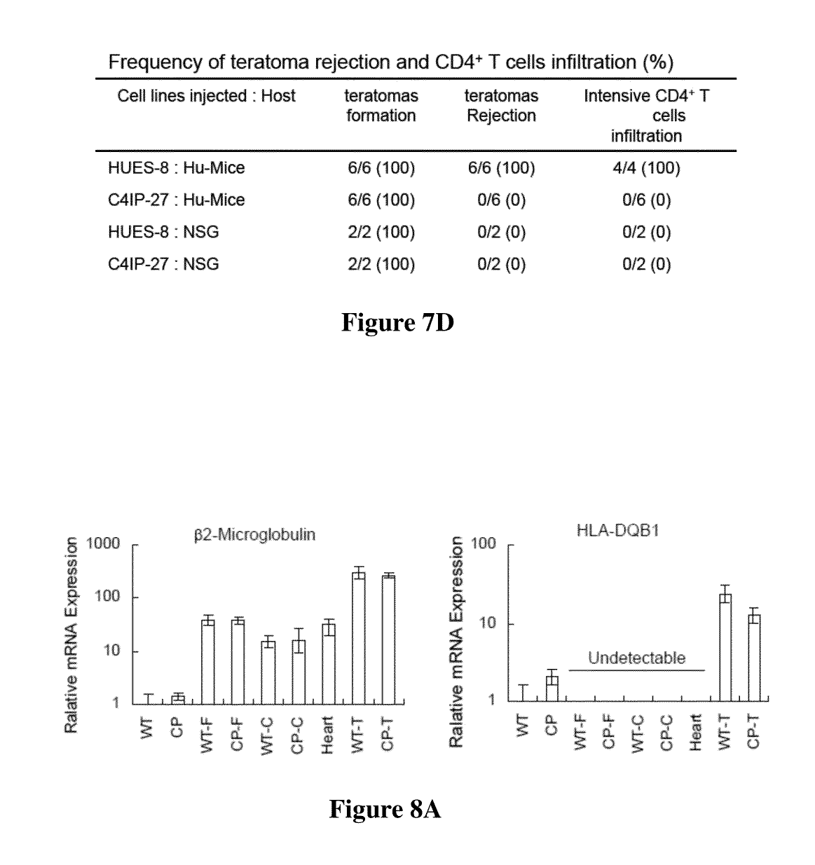

Transient expression of Cre enzyme in the targeted hESCs led to the excision of the selection marker from the genome through LoxP/Cre-mediated deletion, leading to the normal HPRT expression, which is resistant to HAT but sensitive to 6-TG. The expression and secretion of the CTLA4-Ig dimer by CP hESCs was confirmed by Western blotting. When the cells were grown to confluence, the media were changed to DMEM free media. 24 hours later, the media and cells were harvested for western blotting assay. Loading buffer without the reducing agent β-mercaptoethanol was applied to the media samples to check the dimerization status of CTLA4-Ig. In addition, the surface expression of PD-L1 in CP hESCs was confirmed by flow cytometric analysis (FIG. 1C). Without any fixation and permeabilization steps, the cells were sequentially stained with a monoclonal PD-L1 antibody and a PE-conjugated secondary antibody, and then analyzed by FACS. The examination of expression of hESC-specific pluripotency genes and surface markers as well as the capability to form well-differentiated teratomas in SCID mice confirm the pluripotent state of CP hESCs (FIGS. 6A-6C)

Programmed cell death 1 ligand 1 (PD-L1) also known as Cluster of differentiation 274 (CD274) or B7 homolog 1 (B7-H1) is a protein that in humans is encoded by the CD274 gene. PD-L1 is a 40 kDa type 1 transmembrane protein that has been speculated to play a major role in suppressing the immune system during particular events such as pregnancy, tissue allografts, autoimmune disease and other disease states such as hepatitis. Normally the immune system reacts to foreign antigens where there is some accumulation in the lymph nodes or spleen which triggers a proliferation of antigen-specific CD8+ T cell. The formation of PD-1 receptor/PD-L1 ligand complex transmits an inhibitory signal which reduces the proliferation of these CD8+ T cells at the lymph nodes and supplementary to that PD-1 is also able to control the accumulation of foreign antigen specific T cells in the lymph nodes through apoptosis which is further mediated by a lower regulation of the gene Bcl-2. The amino acid sequence of human isoform B of PD-L1 is as follows:

(SEQ ID NO: 1)
mrifavfifm tywhllnapy nkinqrilvv dpvtsehelt
cqaegypkae viwtssdhqv lsgkttttns kreeklfnvt
stlrintttn eifyctfrrl dpeenhtael vipelplahp
pnerthlvil gaillclgva ltfifrlrkg rmmdvkkcgi
qdtnskkqsd thleet

CTLA-4 (Cytotoxic T-Lymphocyte Antigen 4), also known as CD152 (Cluster of differentiation 152), is a protein receptor that down-regulates the immune system. CTLA4 is found on the surface of T cells, which lead the cellular immune attack on antigens. The T cell attack can be turned on by stimulating the CD28 receptor on the T cell. The T cell attack can be turned off by stimulating the CTLA4 receptor, which acts as an “off” switch. In humans, the CTLA4 protein is encoded by the CTLA4 gene. The amino acid sequence of human CTLA-4 is as follows:

(SEQ ID NO: 2)
maclgfqrhk aqlnlatrtw pctllffllf ipvfckamhv
aqpavvlass rgiasfvcey aspgkatevr vtvlrqadsq
vtevcaatym mgneltfldd sictgtssgn qvnitiqglr
amdtglyick velmypppyy lgigngtqiy viakekkpsy
nrglcenapn rarm

CTLA4-Ig is a fusion protein composed of the Fc region of the immunoglobulin IgG1 fused to the extracellular domain of CTLA-4. It is a molecule capable of binding with more avidity to CD80 (B7-1) than to CD86 (B7-2). The amino acid sequence of CTLA4-Ig is as follows:

(SEQ ID NO: 3)
mgvlltqrtllslvlallfpsmasmamhvaqpavvlassrgia
sfvceyaspgkatevrvtvlrqadsqvtevcaatymmgneltf
lddsictgtssgnqvnltiqglramdtglyickvelmypppyy
lgigngtqiyvidpepcpdsdqepkssdkthtsppspapellg
gssvflfppkpkdtlmisrtpevtcvvvdvshedpevkfnwyv
dgvevhnaktkpreeqynstyrvvsyltvlhqdwlngkeykck
vsnkalpapiektiskakgqprepqvytlppsrdeltknqvsl
tclvkgfypsdiavewesngqpennykttppvldsdgsfflys
kltvdksrwqqgnvfscsvmhealhnhytqkslslspgk

As demonstrated herein, a humanized mouse model reconstituted with functional human immune system can mount allogeneic immune response to hESC-derived allograft. There has been limited effort to study the human allogeneic immune responses in vivo due to the lack of physiologically relevant model systems. To address this issue, significant effort has been made to improve humanized mouse models with human immune system during the past three decades (Rongvaux et al., (2013) Human hemato-lymphoid system mice: current use and future potential for medicine, Annu Rev Immunol 31, 635-674; Shultz et al., (2012) Humanized mice for immune system investigation: progress, promise and challenges, Nat Rev Immunol 12, 786-798). Here, the Hu-mouse model has been optimized as a physiologically relevant surrogate to study human allogeneic immune response. In this study, Hu-mice were used to identify a novel combination of immune regulatory molecules to effectively protect hESC-derived grafts from allogeneic immune responses. These findings are instrumental for developing effective immune tolerance strategy without inducing systemic immune suppression.

Standard immune suppressant regiments are effective for preventing immune rejection of allogeneic organs, and their use is justified in the case of terminally ill patients (Selzner et al., (2010) The immunosuppressive pipeline: Meeting unmet needs in liver transplantation, Liver Transplantation 16, 1359-1372). However, the high toxicity of the immunosuppressant regiments, and the increased risk of spontaneous cancer and infection associated with systemic immune suppression, significantly raises the risk/benefit ratio of hESC-based cell therapy. The strategy described herein mitigates these problems by inducing local immune protection of hESC-derived cells without using standard immune suppressants or systemic immune suppression. However, one potential risk of this approach is that it allows the grafted cells with tumorigenic potential or viral infection to escape immune surveillance. One feasible way to address this risk is to introduce a suicidal thymidine kinase (TK) gene into CTLA4-Ig/PD-L1 expression cassette so that the transplanted cells can be effectively eliminated by ganciclovir if needed (Springer and Niculescu-Duvaz, (2000) Prodrug-activating systems in suicide gene therapy, J Clin Invest 105, 1161-1167). In support of this notion, others and we have reported that TK-expressing tumors derived from hESCs could be effectively eliminated in vivo by administration of ganciclovir (Cheng et al., (2012) Protecting against wayward human induced pluripotent stem cells with a suicide gene, Biomaterials 33, 3195-3204; Rong et al., (2012) A scalable approach to prevent teratoma formation of human embryonic stem cells. Journal of Biological Chemistry 287:32338-32345; Schuldiner et al., (2003) Selective ablation of human embryonic stem cells expressing a “suicide” gene, Stem Cells 21, 257-265).

In the context of clinic development, the high targeting efficiency to generate CP hESC lines supports the feasibility to genetically modify any clinic-grade hESCs under the GMP conditions. While the genetic modification of hESCs is a known safety concern in human therapy associated with random integration of the exogenous DNA into the human genome, the knock-in approach employed here should minimize such risk because homologous recombination can be achieved without any apparent random integration of the exogenous DNA (Howden et al., (2011) Genetic correction and analysis of induced pluripotent stem cells from a patient with gyrate atrophy, Proceedings of the National Academy of Sciences 108, 6537-6542; Song et al., (2010) Modeling Disease in Human ESCs Using an Efficient BAC-Based Homologous Recombination System, Cell Stem Cell 6, 80-89). To further address this concern before clinic application of the genetically modified hESCs, the entire genome of CP hESCs will be sequenced to confirm that no other mutations or random insertion of exogenous DNA are introduced during homologous recombination. In summary, the genetic approach described here could be instrumental for developing a safe and scalable approach to effectively protect hESC-derived cells from allogeneic immune rejection without inducing systemic immune suppression.

Accordingly, the present invention provides an expression cassette comprising a promoter functionally linked to a polynucleotide encoding CTLA4-Ig and a polynucleotide encoding PD-L1. Also provided are a vector, such as a bacterial artificial chromosome (BAC)-based targeting vector, that includes the expression cassette.

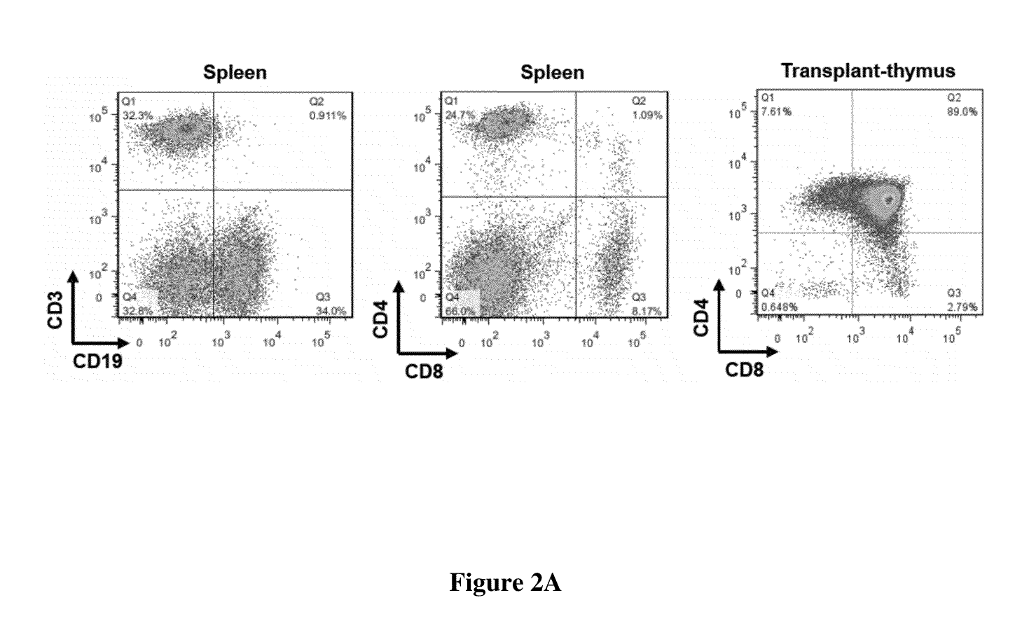

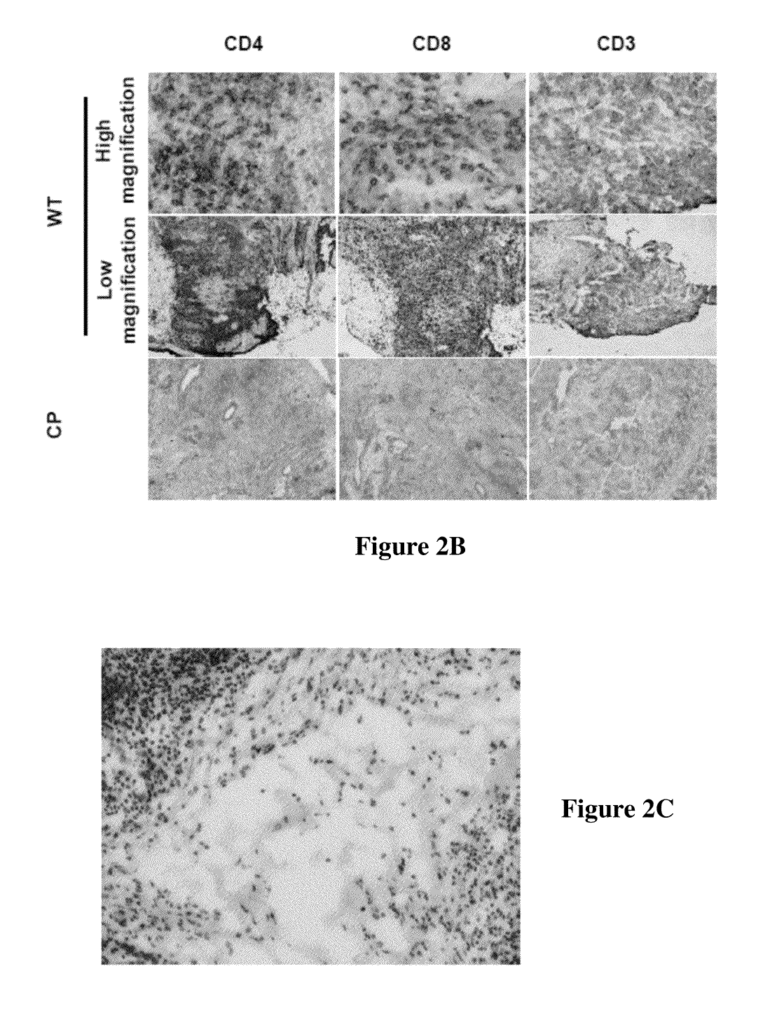

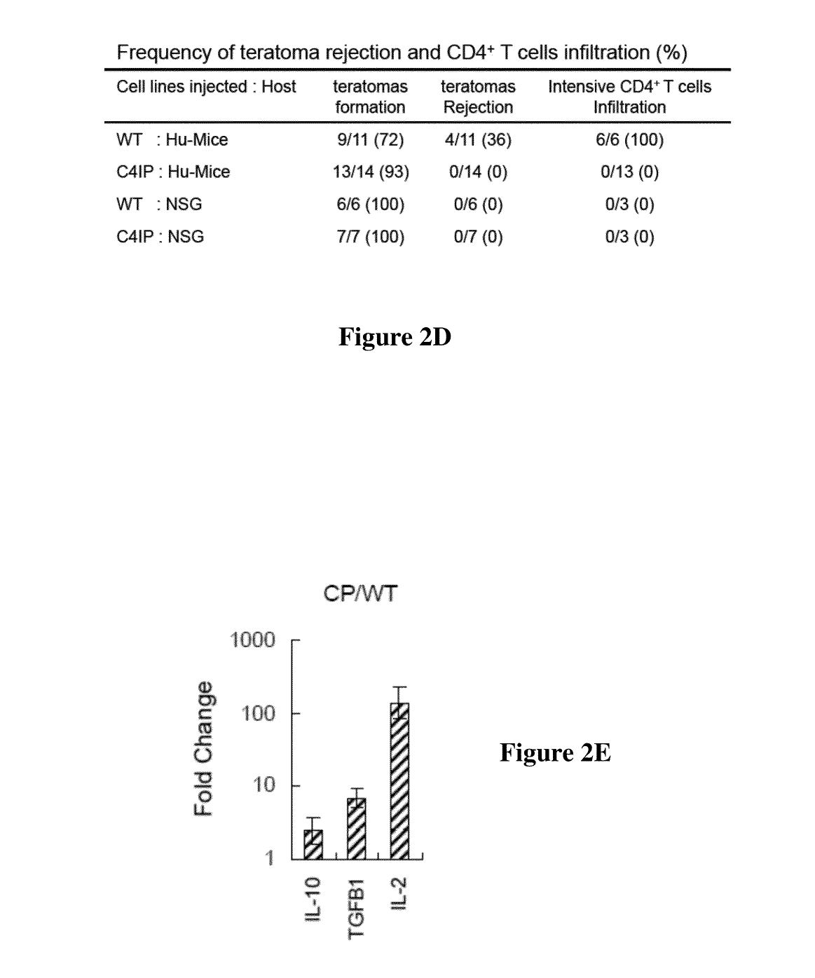

To examine the allogeneic immune rejection of transplanted human cells, humanized mice (Hu-mice) reconstituted with functional human immune system were established by combined transplantation of human fetal thymus/liver tissues and isogenic CD34+ fetal liver cells into the immunodeficient NOD/SCID/IL-2γ^{-/-} (NSG) mice as described (Tonomura, (2008) Antigen-specific human T-cell responses and T cell-dependent production of human antibodies in a humanized mouse model, Blood 111, 4293-4296). Because human immune cells develop de novo in the recipients, the human immune system is thus tolerant of the recipient mouse and does not mediate graft versus host reaction. The risk to develop graft-versus-host disease (GVHD) caused by the residue donor mature T cells in the transplanted fetal thymus is further minimized by one round of freeze/thaw of fetal thymus as recently described (Kalscheuer et al., (2012) A Model for Personalized In Vivo Analysis of Human Immune Responsiveness, Science Translational Medicine 4, 125ra130). The human leukocyte antigen (HLA) typing information of human tissues used to repopulate human immune system in mice and human ESCs is shown in Table 1. The Hu-mice were reconstituted with multi-lineages of human hematopoietic cells, including T cells and B cells (FIG. 2A). In addition, the secondary lymphoid tissue spleen contains well-formed germinal centers, indicating ongoing T-dependent antibody responses (FIG. 2B).

TABLE 1
HLA Typing of Human Tissues and Hues3 and Hues8 ES cells

| Sample | HLA typing | |
|---|---|---|
| human tissue 1# | A2, A11, B51, B57, Cw7, C-, DR7, DR13, (DR52, DR53 blank), DQ9, DQ6, (DQ-, DQ-) | |
| human tissue 2# | A24, A30, B63, B35, Cw4, Cw7, DR-blank, DR18 (DR52, DR52), DQ7, DQ4, (DQ-, DQ-) | |
| Hues3 ES cells | A1, A3, B53, B57, Cw4, Cw6, Dr7, DR14, (DR52, DR53 blank), DQ9, DQ5, (DQ-, DQ-) | |
| Hues8 ES cells | A2, A2, B60, B44, Cw10, Cw5, Dr4, DR-, (DR52, DR53), DQ7, DQ6, (DQ-, DQ-) | |
| | human tissue 1# | human tissue 2# |
| Hues3 ES cells | 7 out of 10 | 9 out of 10 |
| Hues3 ES cells | 6 out of 10 | 7 out of 10 |

To determine whether various lineages of cells derived from CP hESCs were immune rejected by allogeneic human immune response, the capability of hESCs to form teratomas in vivo that contain cells derived from each of the three germ layers was utilized. This allowed for the evaluation of the immunogenicity of various cell types derived from hESCs simultaneously. CP hESCs were implanted subcutaneously into the right flank of a Hu-mouse with an allogeneic immune system. As an internal control, the parental hESCs were implanted into the left flank of the same Hu-mouse. While both CP hESCs and control parental hESCs formed teratomas in Hu-mice, the teratomas formed by parental hESCs regressed rapidly. Single-cell suspensions were stained for the markers of human T cells (CD3, CD4, and CD8), and B cells (CD19) (FIG. 2A).

Hu-mice and NSG mice were subcutaneously injected with WT hESCs, and PD-L1 and CTLA4-Ig expression (C4IP)-hESCs around the left and right hindlegs, respectively. Six to eight weeks after implantation, the mice were euthanized and teratomas examined. The teratomas formed by parental hESCs were much smaller than those formed by CP hESCs, and were constituted primarily of liquid cyst and few cells (FIG. 2A). This conclusion was further supported by the histologic analysis of teratomas formed by parental and CP hESCs, showing few cells in the teratomas remanent formed by parental hESCs, but well-differentiated tissues by CP hESCs in Hu-mice. Because both CP hESCs and parental hESCs could form teratomas of similar size in SCID mice, this ruled out the possibility that the defective teratoma formation by parental hESCs in Hu-mice was not due to impaired pluripotency of the parental hESCs.

As used herein, the term “teratoma” refers to an encapsulated tumor with tissue or organ components resembling normal derivatives of all three germ layers. The tissues of a teratoma, although normal in themselves, may be quite different from surrounding tissues and may be highly disparate; teratomas have been reported to contain hair, teeth, and bone. Usually, however, a teratoma will contain no organs but rather one or more tissues normally found in organs such as the brain, thyroid, liver, and lung. Sometimes, the teratoma has within its capsule one or more fluid-filled cysts; when a large cyst occurs, there is a potential for the teratoma to produce a structure within the cyst that resembles a fetus. Because they are encapsulated, teratomas are usually benign, although several forms of malignant teratoma are known and some of these are common forms of teratoma.

To examine whether the teratomas formed by CP hESCs are protected from the allogeneic immune response, the teratomas formed by parental and CP hESCs in Hu-mince were examined histologically. The expression of PD-L1 was detected by a PD-L1 antibody using immunochemical assay. T cells were identified by CD4, CD8 and CD3 antibodies. The teratoma remnant formed by parental hESCs in HU-mice was consisted mostly of liquid cysts and was extensively infiltrated with human T cells, indicating robust immune rejection (FIGS. 2B-2D). Teratomas with apparent regressing phenotype or containing only liquid-filled cysts without cell mass were classified as rejection. In contrast, the teratomas formed by CP hESCs in Hu-mice were consisted of well-differentiated tissues with greatly reduced T cell infiltration, indicating that cells derived from CP hESCs were protected from the allogeneic immune system (FIGS. 2B-2D). In this context, the number of human T cells infiltrated into parental hESC-derived teratoma is only a few percent of that in CP hESC-derived teratomas formed in the same Hu-mouse, indicating that CP hESC-derived teratomas can be protected from allogeneic immune response without inducing systemic immune suppression. While the percentage of CD4+CD25+Foxp3+ Treg cells in the T cells infiltrating in the teratomas formed by CP hESCs was similar to that in teratomas formed by WT parental hESCs, the T cells purified from the CP-derived teratomas expressed significantly higher levels of immune suppressive cytokines such as IL-10 and TGF-β than those in parental hESC-derived teratomas (FIG. 2E). The majority of teratomas formed by CP hESCs in Hu-mice reached the allowed maximum size by 8 weeks after implantation, some slower growing teratomas formed by CP hESCs were immune protected in Hu-mice for up to three months when they reached the allowed maximum size. Therefore, the greatly reduced T cell infiltration and a local immune suppressive environment contribute to the long-term protection of CP hESC-derived cells from allogeneic immune rejection.

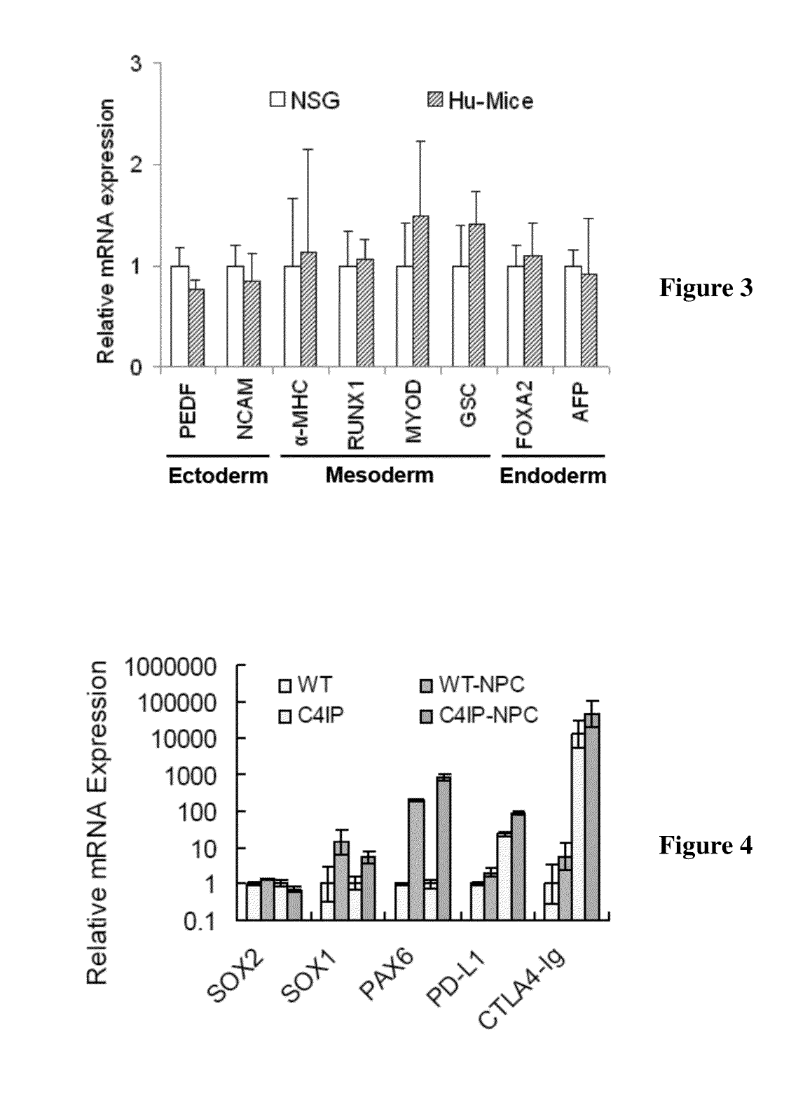

In further support of the conclusion that CP hESC-derived teratomas are protected from allogeneic immune response, the teratomas formed by allogeneic CP hESCs in Hu-mice contained cells derived from each of the three germ layers (FIG. 3). In addition, the comparison of the expression of lineage-specific genes in the teratomas formed by CP hESCs in Hu-mice and NSG mice further confirmed that no specific cell lineages differentiated from CP hESCs were immune rejected. Using the same strategy, the analysis of another independently generated CP HUES-8 hESC line further supports the conclusion that the expression of CTLA4-Ig and PD-L1 by the cells derived from hESCs can protect them from allogeneic immune response (FIGS. 6A-6C). In summary, these data demonstrate that human cells of various lineages differentiated from CP hESCs are protected from allogeneic human immune system.

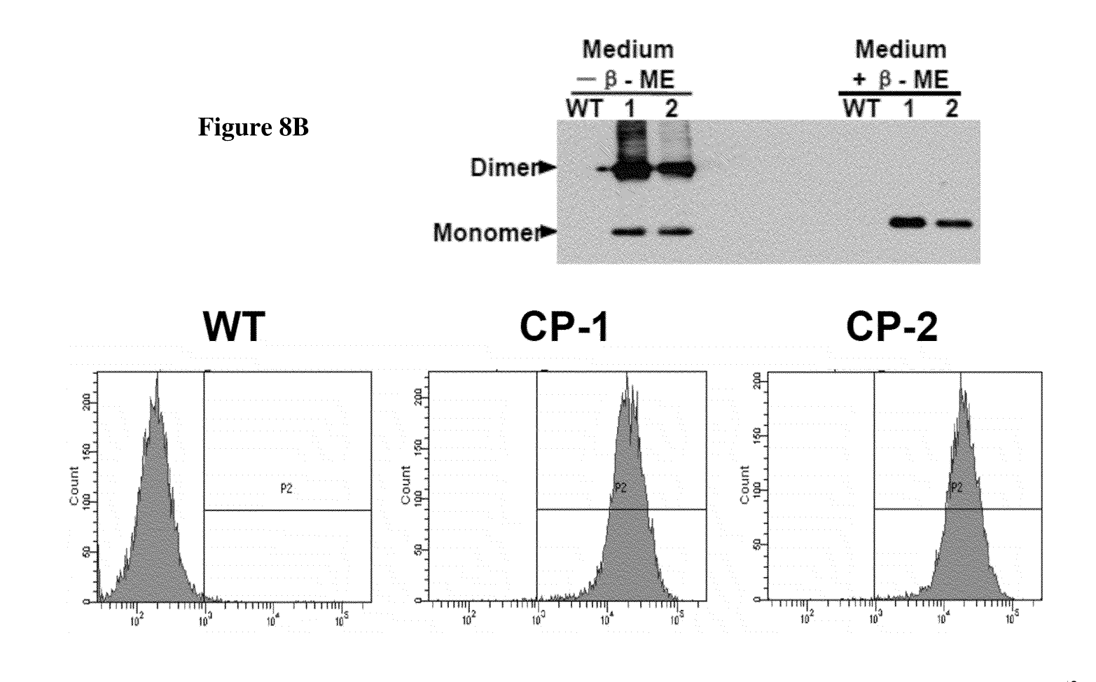

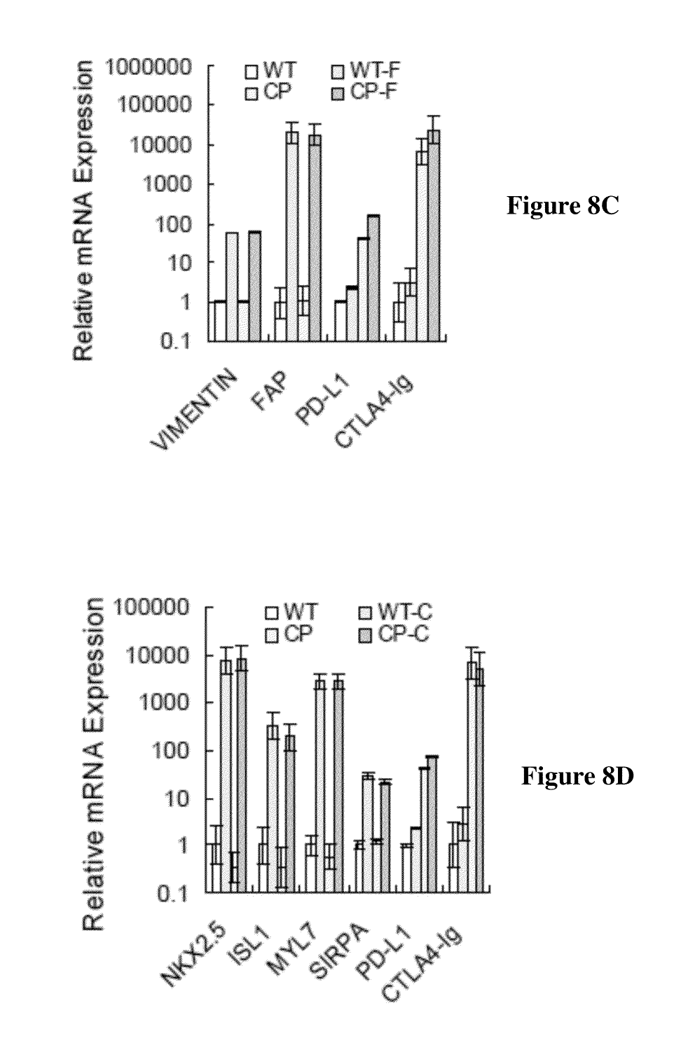

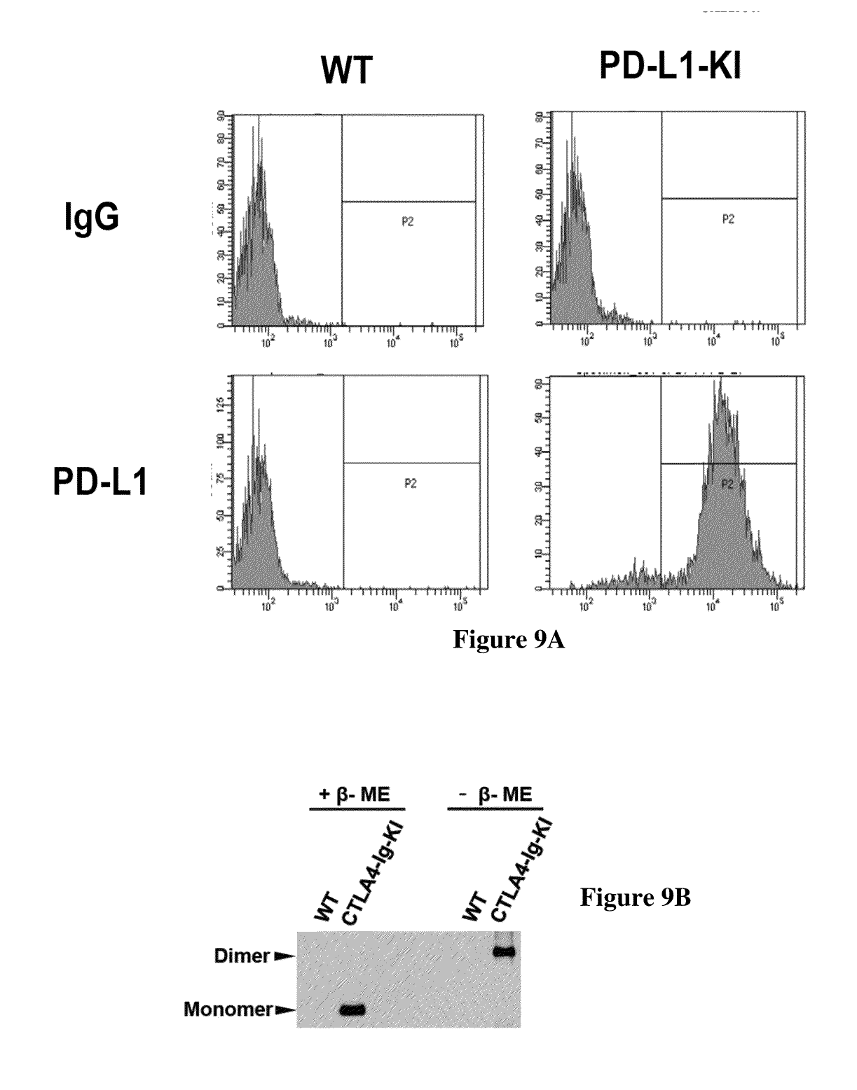

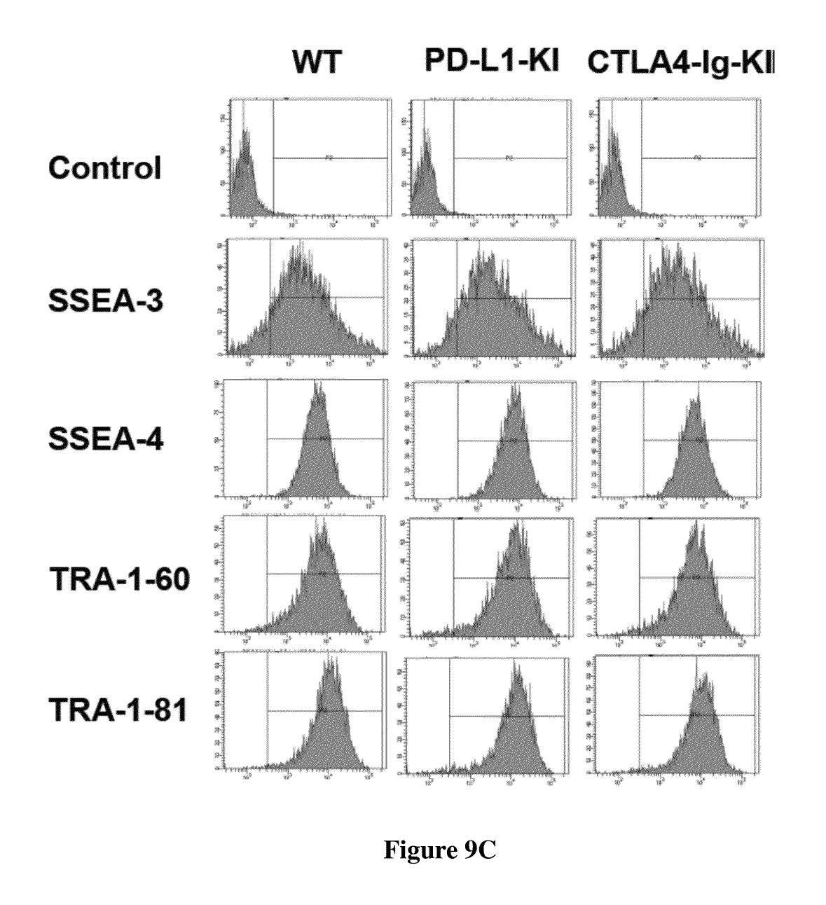

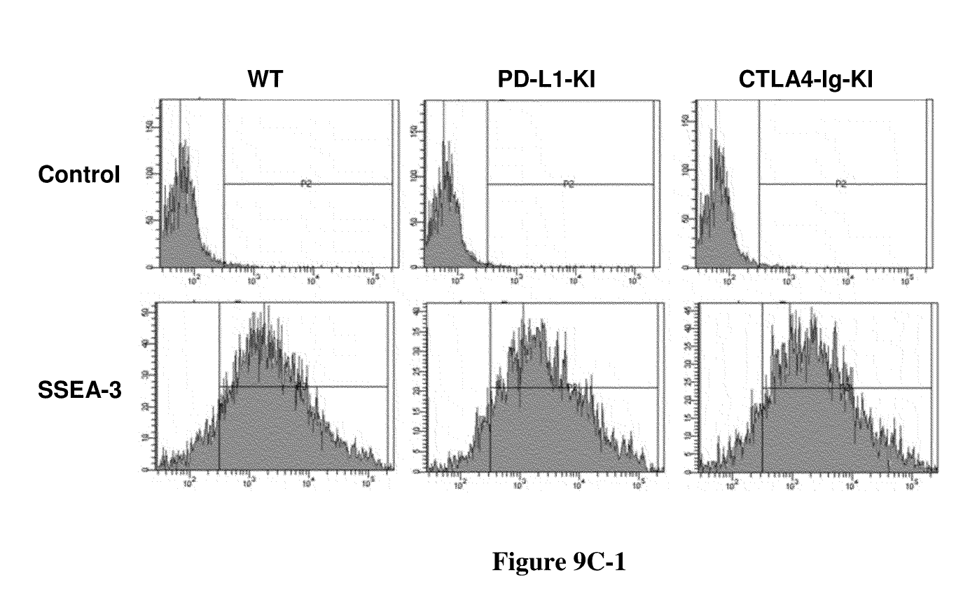

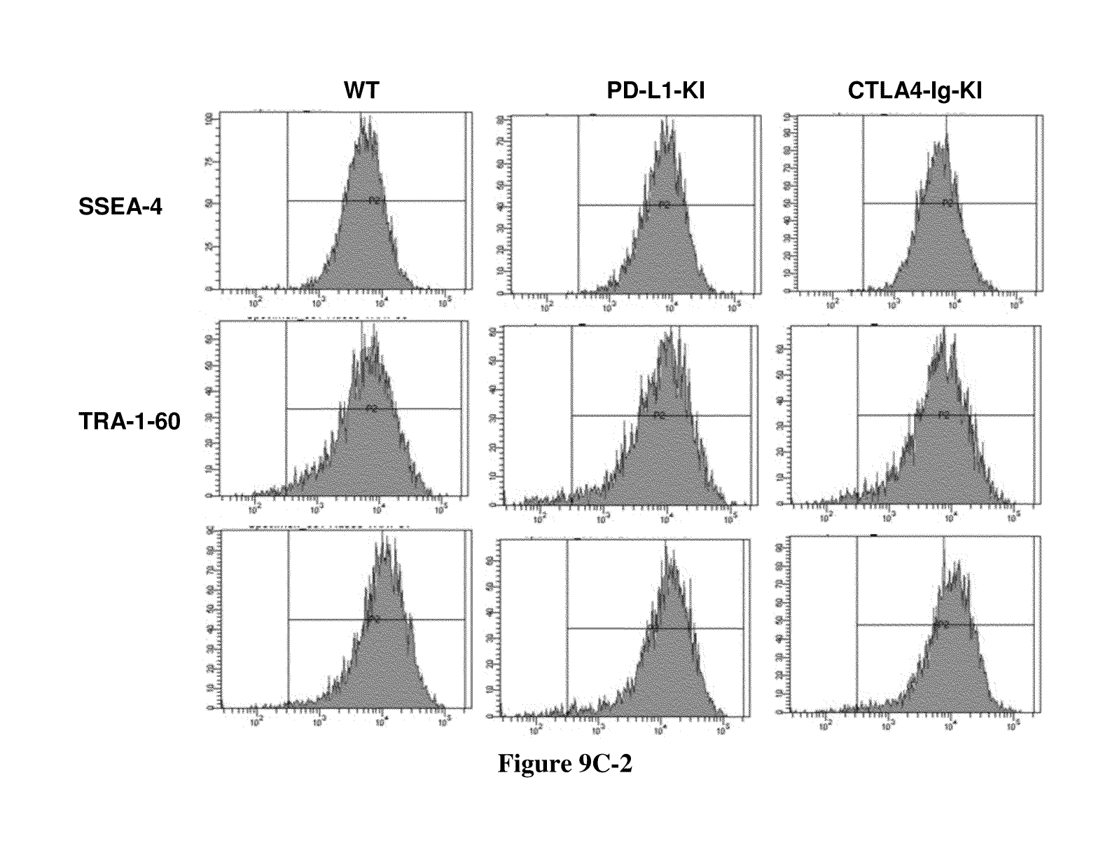

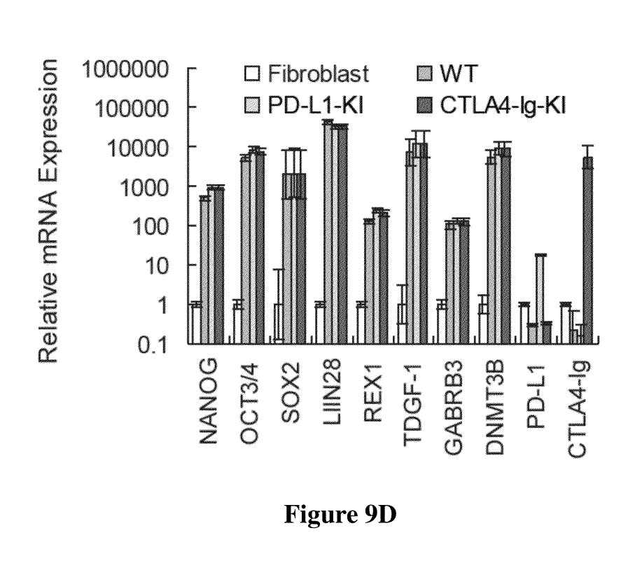

To further confirm that the expression of CTLA4-Ig and PD-L1 does not induce systemic immune response, CP hESCs were implanted into Hu-mice, the teratomas were removed six weeks later and the same Hu-mouse implanted with parental hESCs. Six to eight weeks after implantation, the teratomas formed by parental hESCs were recovered and analyzed for immune rejection, indicating that the parental hESC-derived teratomas were immune rejected with extensive infiltration of T cells (FIG. 8A). In contrast, when parental hESCs mixed with CP hESCs at a ratio of 2:1 were injected into Hu-mice, the resulting teratomas contained cells derived from both WT and CP hESCs without any apparent immune rejection, indicating that the cells derived from CP hESCs can provide local protection of cells derived from parental hESCs from allogeneic rejection (FIG. 8B). These findings further support the conclusion that the local expression of CTLA4-Ig and PD-L1 could achieve immune protection of hESC-derived allografts without inducing systemic immune suppression or immune tolerance.

To further evaluate the immune response to hESC-derived cells, human fibroblasts and cardiomyocytes were derived from CP and control parental hESCs, with their identities confirmed by cell-type-specific gene expression (FIGS. 8C-8D). The CP hESC-derived cells were confirmed for the expression of CTLA4-Ig and PD-L1 (FIG. 8B). The cell membrane expression of PD-L1 in CP-F was determined by flow cytometry. In addition, the increased expression of HLA class I on the differentiated cell types was confirmed by real-time PCR assay (FIG. 8A). Expression of the cardiomyocyte markers α-actinin and Nk×2.5 in cardiomyocytes derived from WT hESCs (WT hESC-C) and CP hESCs (CP hESC-C) were revealed by immunostaining. Likewise, the presence of cTnI+ cardiomyocytes in WT hESC-C and CP hESC-C transplants in Hu-mice was revealed by immunostaining. Human cells were identified by a human nuclei-specific antibody (HuNu). Nuclei were counterstained with DAPI.

Consistent with previous findings (Blomer et al., (2004) Shuttle of lentiviral vectors via transplanted cells in vivo, Gene Ther 12, 67-74; Xu et al., (2008) Highly enriched cardiomyocytes from human embryonic stem cells, Cytotherapy 10, 376-389), the hESC-derived cardiomyocytes could survive for an extended period of time in vivo after being transplanted into the hindleg muscle of NSG mice. For control WT and CP hESC-derived fibroblasts, they were embedded into gel foam and implanted into left and right side of the same Hu-mice subcutaneously. For control and CP hESC-derived cardiomyocytes, they were injected directly into the skeletal muscle of the left and right hind legs of Hu-mice. While significant T cell infiltration was detected in the parental hESC-derived cells transplanted in Hu-mice, greatly reduced T cell infiltration was detected in the graft of CP hESC-derived cells in the same Hu-mice, indicating that CP hESC-derived fibroblasts and cardiomyocytes were also protected from the allogeneic immune response. Therefore, these data demonstrate that somatic cells differentiated from CP hESCs are protected from allogeneic human immune system.

To determine the contribution of CTLA4-Ig and PD-L1 to the immune protection of hESC-derived allografts, the same knock-in strategy was used to introduce the CTLA4-Ig and PD-L1 expression cassette independently into the HPRT locus of hESCs. Once confirmed for their pluripotency and the expression of CTLA4-Ig or PD-L1, the parental and knock-in hESCs were transplanted subcutaneously into the left and right flank of Hu-mice. Thus, WT hESCs were implanted into the Hu-mice that were previously implanted with CP hESCs followed by surgical removal of CP hESC-derived teratomas. Likewise, a mixture of CP hESCs and WT hESCs (ratio 1:2) was implanted into the Hu-mice.

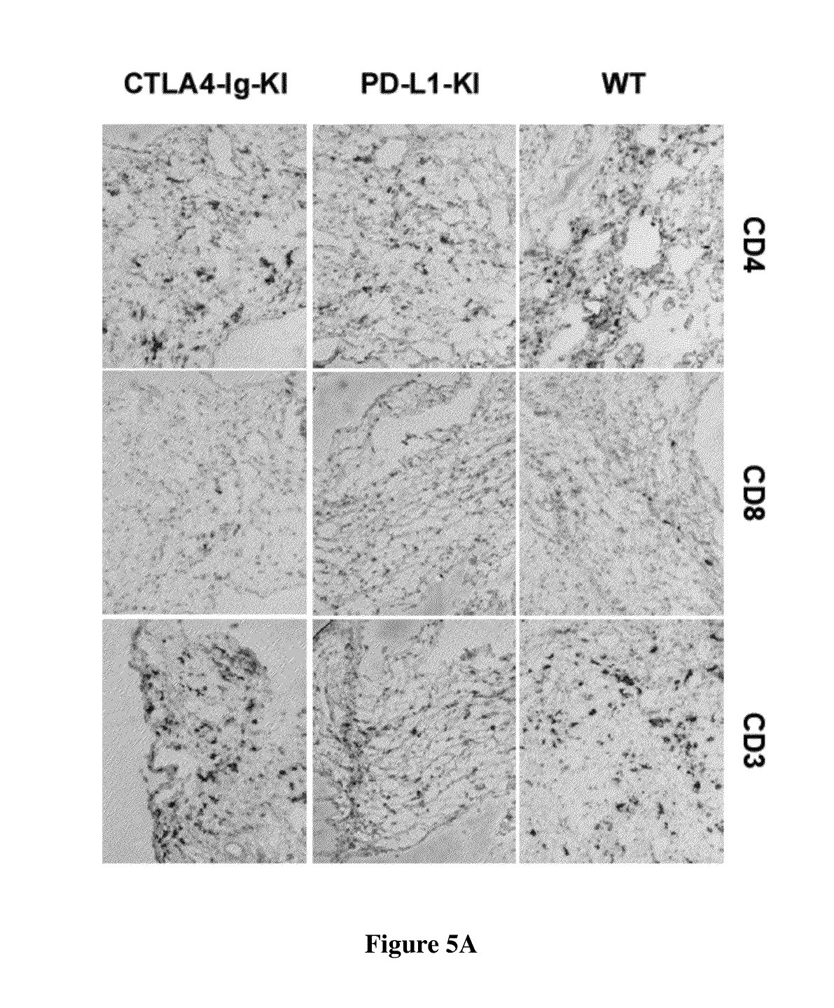

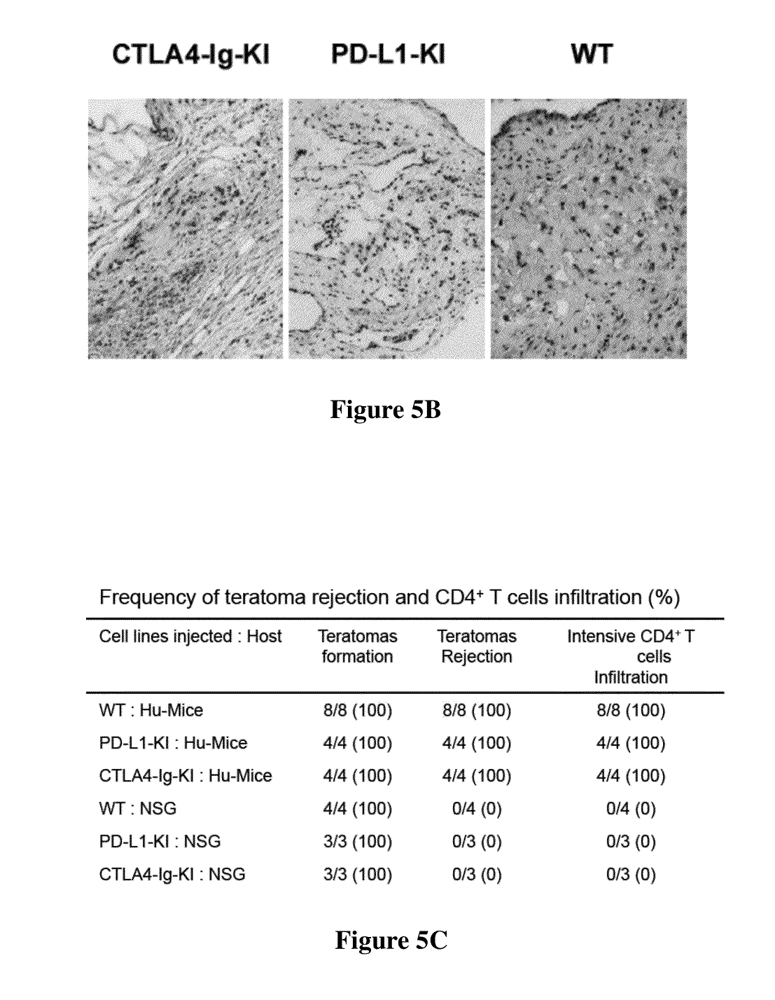

The teratomas derived from WT hESCs were extensively infiltrated with human T cells six to eight weeks after implantation. In contrast to the teratomas formed by CP hESCs that are protected from allogeneic immune system in Hu-mice, teratomas formed by knock-in hESCs expressing only CTLA4-Ig or PD-L1 were robustly immune rejected in Hu-mice, similarly to the teratomas formed by parental hESCs (FIGS. 5A-5C). Therefore, CTLA4-Ig and PD-L1 must work together to protect the transplanted cells from the allogeneic immune responses.

Accordingly, in another aspect, the present invention provides a method of preventing allogeneic rejection of human embryonic stem cells (hESCs) and/or hESC-derived cells in a host organism. In some embodiments, the host organism is a human. In other embodiments, the host organism is a non-human mammal. The method includes administering to the host organism hESC-derived cells and a vector, where the vector includes a polynucleotide encoding CTLA4-Ig, a polynucleotide encoding PD-L1, and a promoter operably linked thereto. As such, allowing expression of CTLA4-Ig and PD-L1 in the host organism prevents allogeneic rejection of the hESC-derived cells. In various embodiments, the method may further include obtaining hESCs and transfecting the cells with a vector encoding CTLA4-Ig and PD-L1. The resulting hESC-derived cells may then be administered to the host organism.

In yet another aspect, the present invention provides a method of preventing allogeneic rejection of allogeneic cells in a host organism. In some embodiments, the host organism is a human. In other embodiments, the host organism is a non-human mammal. The method includes administering to the host organism in need thereof a therapeutically effect amount of CTLA4-Ig and PD-L1. In various embodiments, the CTLA4-Ig and PD-L1 may be administered alone or in combination with hESC-derived cells or other allogeneic cells, such that allogeneic rejection of the hESC-derived cells, or other allogeneic cells, is reduced and/or inhibited.

The methods of the present invention may further include the step of bringing the active ingredient(s) (e.g., CTLA4-Ig and PD-L1) into association with a pharmaceutically acceptable carrier, which constitutes one or more accessory ingredients. As such, the invention also provides pharmaceutical compositions for use in preventing allogeneic rejection of allogeneic cells. In one embodiment, the composition includes as the active constituent a therapeutically effective amount of CTLA4-Ig and PD-L1 as discussed above, together with a pharmaceutically acceptable carrier, diluent of excipient. In another embodiment, the allogeneic cells are derived from hESCs.

Pharmaceutically acceptable carriers useful for formulating a composition for administration to a subject are well known in the art and include, for example, aqueous solutions such as water or physiologically buffered saline or other solvents or vehicles such as glycols, glycerol, oils such as olive oil or injectable organic esters. A pharmaceutically acceptable carrier can contain physiologically acceptable compounds that act, for example, to stabilize or to increase the absorption of the conjugate. Such physiologically acceptable compounds include, for example, carbohydrates, such as glucose, sucrose or dextrans, antioxidants, such as ascorbic acid or glutathione, chelating agents, low molecular weight proteins or other stabilizers or excipients. One skilled in the art would know that the choice of a pharmaceutically acceptable carrier, including a physiologically acceptable compound, depends, for example, on the physico-chemical characteristics of the therapeutic agent and on the route of administration of the composition, which can be, for example, orally or parenterally such as intravenously, and by injection, intubation, or other such method known in the art. The pharmaceutical composition also can contain a second (or more) compound(s) such as a diagnostic reagent, nutritional substance, toxin, or therapeutic agent, for example, a cancer chemotherapeutic agent and/or vitamin(s).

The total amount of a compound or composition to be administered in practicing a method of the invention can be administered to a subject as a single dose, either as a bolus or by infusion over a relatively short period of time, or can be administered using a fractionated treatment protocol, in which multiple doses are administered over a prolonged period of time (e.g., once daily, twice daily, etc.). One skilled in the art would know that the amount of CTLA4-Ig and PD-L1, or functional fragments thereof to prevent allogeneic rejection of allogeneic cells in a subject depends on many factors including the age and general health of the subject as well as the route of administration and the number of treatments to be administered. In view of these factors, the skilled artisan would adjust the particular dose as necessary. In general, the formulation of the pharmaceutical composition and the routes and frequency of administration are determined, initially, using Phase I and Phase II clinical trials.

The following examples are intended to illustrate but not limit the invention.

### Example 1

### Construction of BAC-Based Targeting Vector

The HPRT1 BAC clone RP11-671P4 was purchased from Invitrogen and the targeting vector was constructed by recombineering as previously described (Rong et al., (2012) A scalable approach to prevent teratoma formation of human embryonic stem cells. Journal of Biological Chemistry 287:32338-32345; Song et al., (2010) Modeling Disease in Human ESCs Using an Efficient BAC-Based Homologous Recombination System. Cell Stem Cell 6, 80-89). Briefly, the pCAG/CTLA4-Ig/IRES/PD-L1/polyA expression cassette was inserted 600 bp downstream of the HPRT1 stop codon. The Loxp-flanked selection cassette pCAG/Neo/IRES/Puro/polyA was inserted between the HPRT1 stop codon and its endogenous polyA site. The Cre-mediated deletion of the selection cassette will restore the normal expression of HPRT.

Cell Culture—

The hESC lines, HUES-3 and HUES-8, were cultured on mouse embryonic fibroblast feeder layer in Knockout Dulbecco's modified Eagle's medium (DMEM) supplemented with 10% knockout serum replacement (KOSR), 10% plasmanate, 0.1 mM nonessential amino acids, 2 mM Glutamax, 1% penicillin/streptomycin, 10 ng/ml bFGF, and 55 μM β-mercaptoethanol. HUES hESCs were dissociated with TryLE and passaged on feeder with 1:4-1:6 dilution. To determine the secretion of CTLA4-Ig, the cells were grown to confluence and the media changed to serum-free DMEM media. Twenty-four hours later, the media and cells were harvested for Western blotting assay. All tissue culture reagents were purchased from Invitrogen unless indicated otherwise.

HAT and 6-TG Cytotoxicity Assays—

hESCs were plated on 12-well plate, and 24 hrs later, media were replaced with that containing 100 μM hypoxanthine/0.4 μM aminopterin/16 μM thymidine (HAT, Sigma), or 1 mM 6-thioguanine (6-TG, Sigma), or were mock treated. After 3 days of treatment, the cells were stained with an alkaline phosphatase detection kit according to the manufacturer's instruction (Millipore).

Flow Cytometric Analysis—

The flow cytometric analysis of the surface expression of hESC-specific markers, TRA 1-61, TRA 1-81, SSEA3 and SSEA4, was analyzed as described previously (Rong et al., (2012) A scalable approach to prevent teratoma formation of human embryonic stem cells. Journal of Biological Chemistry 287:32338-32345). Briefly, 5×10⁵ cells were washed with PBS, stained for 1 hr at room temperature with primary antibody. After being washed twice with PBS, the cells were stained with a fluorescein isothiocyanate (FITC)/phycoerythrin (PE)-conjugated secondary antibody for 30 min at room temperature and analyzed by a BD LSR-II machine using FACS Diva software (Becton Dickinson). For flow cytometric analysis of spleen and thymus in humanized mouse, a single cell suspension was mashed through 40 μM cell strainers and washed with PBS. Red blood cells in spleen samples were removed with ACK lysis buffer. The primary antibodies used were: anti-PD-L1 antibody (29E.2A3, Biolegend), PE-anti-hCD3 (eBioScience), FITC-anti-hCD19 (eBioScience), PE-anti-hCD4 (BD Pharmingen), and FITC-anti-hCD8 (BD Pharmingen).

Western Blotting Assay—

To analyze the expression of CTLA4-Ig by CP hESCs and their derivatives, the cells were grown to confluence, the media replaced with DMEM basal media. Twenty-four hrs later, two aliquots of the media were harvested, boiled in 1× loading buffer with or without β-mercaptoethanol, and fractionated by SDS-PAGE and transferred to nitrocellulose membrane. After blocked with 10% milk for 45 min at room temperature, the membrane was probed with a horseradish peroxidase (HRP)-conjugated goat anti-human IgG-Fc antibody (Bethyl) overnight at 4° C., and developed with an enhanced chemilluminescent substrate (Pierce).

Humanized Mice with Functional Human Immune System—

After conditioned with sublethal (2.25 Gy) total body irradiation, NOD.Cg-Prkdc^{scid} Il2rg^{tmlwil}/SzJ (NOD/SCID/γc^{-/-} or NSG, The Jackson Laboratory) mice of 6-10 weeks of age were transplanted with human fetal thymic tissue piece (previously frozen about 1 mm³) under the kidney capsule, and intravenously transfused 1-5×10⁵ human CD34⁺ fetal liver cells as previously described (Lan et al., (2006) Reconstitution of a functional human immune system in immunodeficient mice through combined human fetal thymus/liver and CD34+ cell transplantation. Blood 108, 487-492). Human fetal tissues of gestational age of 17-20 weeks were obtained from Advanced Bioscience Resource (Alameda, Calif.). Human CD34⁺ cells were isolated by a magnetic-activated cell sorter separation system using anti-CD34 microbeads (Miltenyi Biotec).

Immunohistochemical and Histologic Analysis—

Teratoma was fixed in 10% (w/v) buffered formalin, embedded in paraffin and sectioned as previously described (Zhao et al., (2011) Immunogenicity of induced pluripotent stem cells. Nature 474, 212-215). Briefly, the sections were stained with hematoxylin and eosin for histological assessment, and stained with anti-NeuN (Millipore), anti-C-peptide (Abcam) and anti-karetin (Millipore) antibodies for immunohistochemistry analysis. For frozen sections, samples were frozen in optimal cutting temperature (OCT) compound and sectioned. Teratoma sections were fixed in cold acetone for 10 min, sequentially blocked with 0.03% H₂O₂ for 30 min, 0.1% avidin for 15 min, 0.01% biotin for 15 min and 1% BSA for 30 min. Sections were incubated with primary antibody overnight at 4° C., biotinylated secondary antibody for 30 min, HRP streptavidin for 30 min, and developed with aminoethyl carbazole (AEC) substrate solution. After counterstaining with hematoxylin, sections were mounted in aqueous gel Vectamount (Vector). Images were captured with an Olympus MVX10 Microscope or an Olympus FluoView 1000 Confocal Microscope. Antibodies used: anti-CD3 antibody (eBioScience), anti-CD4, anti-CD8 antibodies (BD Pharmingen), anti-PD-L1 antibody (Biolegend), anti-human nuclei antibody (Millipore), anti-α-actin antibody (Sigma), anti-cardiac Troponin I antibody (Epitomics).

Differentiation of hESCs into Cardiomyocytes—

A small molecule-driven cardiomyocyte differentiation protocol was used (Lian et al., (2012) Robust cardiomyocyte differentiation from human pluripotent stem cells via temporal modulation of canonical Wnt signaling. Proceedings of the National Academy of Sciences 109, E1848,ÄìE1857). hESCs were maintained on matrigel-coated plate. When the culture reached confluence, cells were plated with GSK3 inhibitor CHIR99021 in RPMI/B27-insulin (without insulin) medium for 24 hr (day 0 to day 1), and subsequently changed to RPMI/B27-insulin. On day 3, the cells were exposed to IWP4 (Stemgent) for 2 days. From day 7, the cells were cultured in RPMI/B27 medium with medium change every 3 days.

Transplantation of hESC-Derived Fibroblasts and Cardiomyocytes—

Derivation of human fibroblasts from the teratomas formed by hESCs was performed as previously described (Rong et al., (2012) A scalable approach to prevent teratoma formation of human embryonic stem cells. Journal of Biological Chemistry 287:32338-32345). Human fibroblasts were harvested and washed with PBS. About 20 μl of fibroblast suspension (1×10⁷ cells) was loaded onto 6-7 mm diameter×1.5 mm thick gelatin foam (Gelfoam, Pfizer) discs that were pre-wetted with DMEM basal media. The cells were overlaid with 15-25 μl Matrigel (BD Bioscience) and implanted subcutaneously with cells facing the dermis. hESC-derived cardiomyoctes were harvested, washed with PBS, suspended in PBS with 50% matrigel, and intramuscularly injected into the hind leg gastrocnemius with a 20G needle.

Quantitative Real Time PCR Analysis—

The total RNA was purified from hESCs, human T cells isolated from teratoma, hESC-derived fibroblasts and cardiomyocytes as previously described (Rong et al., (2012) A scalable approach to prevent teratoma formation of human embryonic stem cells. Journal of Biological Chemistry 287:32338-32345) (FIG. 4). The primers used for hESC markers (NANOG, OCT4, SOX2, LIN28, REX1, TDGF-1, GABRB3 and DNMT3B), fibroblast markers (VIMENTIN and FAP) and internal control (GAPDH) were previously described (Rong et al., 2012). The primers used for cardiomyocyte markers (NKX2.5, ISL1, MYL7 and SIRPA) were previously described (Dubois et al., (2011) SIRPA is a specific cell-surface marker for isolating cardiomyocytes derived from human pluripotent stem cells. Nat Biotech 29, 1011-1018). Other primers used are listed in Table 2.

TABLE 2
Primers used in real-time PCR assays

| | Primers (5′-3′) | |
|---|---|---|
| | Forward | Reverse |
| PD-L1 | GTGGTGCCGACTACAAGCGA | TTTGGAGGATGTGCCAGAGGT |
| | (SEQ ID NO: 4) | (SEQ ID NO: 5) |
| CTLA4-Ig | TACCCACCGCCATACTACCT | CTCAGGGTCTTCGTGGCTCA |
| | (SEQ ID NO: 6) | (SEQ ID NO: 7) |
| PEDF | ATCGAGTACATCTTCAAGCCAT | CTTTCTTTGGTCTGCATTCACA |
| | (SEQ ID NO: 8) | (SEQ ID NO: 9) |
| NCAM | TCTATAACGCCAACATCGACGA | TTGGCGCATTCTTGAACATG |
| | (SEQ ID NO: 10) | (SEQ ID NO: 11) |
| α-MHC | AGTGCTTCGTGCCCGATGAC | TGCTGCAACACCTGTCCT |
| | (SEQ ID NO: 12) | (SEQ ID NO: 13) |
| RUNX1 | CCCTAGGGGATGTTCCAGAT | TGAAGCTTTTCCCTCTTCCA |
| | (SEQ ID NO: 14) | (SEQ ID NO: 15) |
| MYOD | CCGCCTGAGCAAAGTAAATGA | GCAACCGCTGGTTTGGATT |
| | (SEQ ID NO: 16) | (SEQ ID NO: 17) |
| GSC | GAGGAGAAAGTGGAGGTCTGGTT | CTCTGATGAGGACCGCTTCTG |
| | (SEQ ID NO: 18) | (SEQ ID NO: 19) |
| FOXA2 | GGGAGCGGTGAAGATGGA | TCATGTTGCTCACGGAGGAGTA |
| | (SEQ ID NO: 20) | (SEQ ID NO: 21) |
| AFP | AGCTTGGTGGTGGATGAAAC | CCCTCTTCAGCAAAGCAGAC |
| | (SEQ ID NO: 22) | (SEQ ID NO: 23) |
| IL-10 | AAGGCGCATGTGAACTCCC | ACGGCCTTGCTCTTGTTTTC |
| | (SEQ ID NO: 24) | (SEQ ID NO: 25) |
| TGFB1 | CCCAGCATCTGCAAAGCTC | GTCAATGTACAGCTGCCGCA |
| | (SEQ ID NO: 26) | (SEQ ID NO: 27) |
| IL-2 | CAACTGGAGCATTTACTGCTG | TCAGTTCTGTGGCCTTCTTGG |
| | (SEQ ID NO: 28) | (SEQ ID NO: 29) |
| B2- | CTGGGTTTCATCCATCCGA | TGCGGCATCTTCAAACCTC |
| Microglobulin | (SEQ ID NO: 30) | (SEQ ID NO: 31) |
| HLA-DQB1 | TGGAACAGCCAGAAGGAAG | AGCAGGTTGTGGTGGTTGA |
| | (SEQ ID NO: 32) | (SEQ ID NO: 33) |

### Example 2

### Preventing Allogeneic Rejection of hESC-Derived Cells or Other Allogeneic Cells by Genetic Modification

As described herein, hESCs will be obtained and transfected with a vector encoding CTLA4-Ig and PD-L1. The progeny of such transfected hESCs will therefore express CTLA4-Ig and PD-L1, thereby preventing allogeneic rejection of the cells upon administration to a human subject. The genetically modified hESC-derived cells will then be administered to a subject in need thereof.

### Example 3

### Preventing Allogeneic Rejection of hESC-Derived Cells by Protein Administration

As described herein, allogeneic rejection of administered hESC-derived cells will be avoided by co-administering the hESC-derived cells with CTLA4-Ig and membrane-bound PD-L1 to a human subject.

Although the invention has been described with reference to the above example, it will be understood that modifications and variations are encompassed within the spirit and scope of the invention. This application is intended to cover any variations, uses, or adaptations of the disclosure following, in general, the disclosed principles and including such departures from the disclosure as come within known or customary practice within the art to which the disclosure pertains and as may be applied to the essential features hereinbefore set forth. Accordingly, the invention is limited only by the following claims.

What is claimed is:

1. A method of preventing allogeneic rejection of allogeneic cells in a subject comprising administering to the subject allogeneic cells genetically modified by a vector comprising a polynucleotide encoding CTLA4-Ig, a polynucleotide encoding PD-L1, and a promoter, wherein allowing expression of CTLA4-Ig and PD-L1 in the subject prevents allogeneic rejection of the allogeneic cells.

2. The method of claim 1, wherein the host subject is mammalian.

3. The method of claim 2, wherein the subject is human.

4. The method of claim 1, wherein the allogeneic cells are derived from human embryonic stem cells (hESCs).

5. The method of claim 4, wherein the allogeneic cells are brown adipocytes, cardiomyocytes, pancreatic beta cells, cartilage or bone-forming cells, or vascular cells.

6. An expression cassette comprising a promoter functionally linked to a polynucleotide encoding CTLA4-Ig and a polynucleotide encoding PD-L1.

7. A vector comprising the expression cassette according to claim 6.

8. The vector of claim 7, wherein the vector is a bacterial artificial chromosome (BAC)-based targeting vector.

9. A mammalian host cell containing the expression vector according to claim 7.

10. A method of preventing allogeneic rejection of allogeneic cells in a subject in need thereof comprising administering to the subject an effective amount of CTLA4-Ig and PD-L1, thereby preventing allogeneic rejection of allogeneic cells in the subject.

11. The method of claim 10, further comprising administering to the subject allogeneic cells.

12. The method of claim 11, wherein the allogeneic cells are cells are derived from hESCs.

13. The method of claim 11, wherein the subject is mammalian.

14. The method of claim 13, wherein the subject is human.

15. The method of claim 14, wherein the cells are brown adipocytes, cardiomyocytes, pancreatic beta cells, cartilage or bone-forming cells, or vascular cells.

16. A pharmaceutical composition comprising CTLA4-Ig, PD-L1, and a pharmaceutically acceptable carrier.
